(19) United States
(12) Patent Application Publication
(10) Pub. No.: US 2017/0283404 A1
(43) Pub. Date: Oct. 5, 2017

(54) CRYSTALLINE FORMS OF 2-(4-(4-ETHOXY-6-OXO-1,6-DIHYDROPYRIDIN-3-YL)-2-FLUOROPHENYL)-N-(5-(1,1,1-TRIFLUORO-2-METHYLPROPAN-2-YL)ISOXAZOL-3-YL)ACETAMIDE

(71) Applicant: GLAXOSMITHKLINE INTELLECTUAL PROPERTY DEVELOPMENT LIMITED, Brentford, Middlesex (GB)

(72) Inventors: Mui CHEUNG, King of Prussia, PA (US); William M. CLARK, King of Prussia, PA (US); Hilary Schenck EIDAM, King of Prussia, PA (US); Kimberly Anne LAMEY, Collegeville, PA (US); James V. THOMAS, Collegeville, PA (US)

(73) Assignee: GlaxoSmithKline Intellectual Property Development Limited, Brentford, Middlesex (GB)

(21) Appl. No.: 15/509,255

(22) PCT Filed: Sep. 4, 2015
(86) PCT No.: PCT/IB15/56766

§ 371 Date: Mar. 7, 2017

Related U.S. Application Data
(60) Provisional application No. 62/047,141, filed on Sep. 8, 2014.

(51) Int. Cl. C07D 413/12

(52) U.S. Cl. CPC C07D 413/12; C07B 2200/13

(57) ABSTRACT

Disclosed are novel crystalline forms of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide and pharmaceutical compositions containing the same. Also disclosed are processes for the preparation thereof and methods for use thereof.

### BACKGROUND OF THE INVENTION

In the pursuit of a developable form of a solid, orally-administered pharmaceutical compound, a number of specific features are sought. Although an amorphous form of a pharmaceutical compound may be developed, compounds having high crystallinity are generally preferred.

International Patent Application Number PCT/IB2014/059817 describes a series of compounds which are indicated as inhibitors of the Rearranged during Transfection (RET) kinase, and which are indicated as being useful in the treatment of RET-mediated disorders. Specifically disclosed in that application is the compound 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (hereinafter “Compound A”). Identification of a stable, crystalline form of such compound with suitable properties for oral administration would be highly desirable for the treatment of RET-mediated diseases.

### SUMMARY OF THE INVENTION

The present invention relates to novel crystalline forms of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide. The compound of the invention is represented by Formula (I):

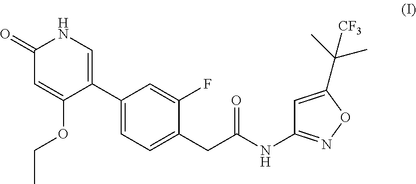 [SMILES: CCOc1cc(=O)[nH]cc1-c1ccc(CC(=O)Nc2cc(C(C)(C)C(F)(F)F)on2)c(F)c1]

The compound of this invention is useful for inhibiting Rearranged during Transfection (RET) kinase, and for the normalization of gastrointestinal sensitivity, motility and/or secretion and/or abdominal disorders or diseases and/or treatment related to diseases related to RET dysfunction or where modulation of RET activity may have therapeutic benefit including but not limited to all classifications of irritable bowel syndrome (IBS) including diarrhea-predominant, constipation-predominant or alternating stool pattern, functional bloating, functional constipation, functional diarrhea, unspecified functional bowel disorder, functional abdominal pain syndrome, chronic idiopathic constipation, functional esophageal disorders, functional gastroduodenal disorders, functional anorectal pain, inflammatory bowel disease, proliferative diseases such as non-small cell lung cancer, hepatocellular carcinoma, colorectal cancer, medullary thyroid cancer, follicular thyroid cancer, anaplastic thyroid cancer, papillary thyroid cancer, brain tumors, peritoneal cavity cancer, solid tumors, other lung cancer, head and neck cancer, gliomas, neuroblastomas, Von Hippel-Lindau Syndrome and kidney tumors, breast cancer, fallopian tube cancer, ovarian cancer, transitional cell cancer, prostate cancer, cancer of the esophagus and gastroesophageal junction, biliary cancer and adenocarcinoma, and any malignancy with increased RET kinase activity.

### BRIEF DESCRIPTION OF THE DRAWINGS

FIG. 1 shows an X-ray powder diffraction pattern of Compound A—Monohydrate.

FIG. 2 shows a Raman spectrum of Compound A—Monohydrate.

FIG. 3 shows a differential scanning calorimetry trace of Compound A—Monohydrate.

FIG. 4 shows a thermogravimetric analysis trace of Compound A—Monohydrate.

FIG. 5 shows an X-ray powder diffraction pattern of Compound A—Non-solvated Form 1.

FIG. 6 shows a Raman spectrum of Compound A—Non-solvated Form 1.

FIG. 7 shows a differential scanning calorimetry trace of Compound A—Non-solvated Form 1.

FIG. 8 shows a thermogravimetric analysis trace of Compound A—Non-solvated Form 1.

FIG. 9 shows an X-ray powder diffraction pattern of Compound A—Non-solvated Form 2.

FIG. 10 shows a Raman spectrum of Compound A—Non-solvated Form 2.

FIG. 11 shows a differential scanning calorimetry trace of Compound A—Non-solvated Form 2.

FIG. 12 shows an X-ray powder diffraction pattern of Compound A—Non-solvated Form 3.

FIG. 13 shows a Raman spectrum of Compound A—Non-solvated Form 3.

FIG. 14 shows a differential scanning calorimetry trace of Compound A—Non-solvated Form 3.

FIG. 15 shows a thermogravimetric analysis trace of Compound A—Non-solvated Form 3.

### DETAILED DESCRIPTION OF THE INVENTION

The present invention is directed to crystalline forms of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide.

In some embodiments, a crystalline form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Monohydrate) is characterized by an X-ray powder diffraction (XRPD) pattern comprising at least nine diffraction angles, when measured using Cu K_{α} radiation, selected from a group consisting of about 10.1, 10.7, 11.5, 13.2, 13.9, 14.3, 16.7, 17.1, 17.6, 18.3, 18.4, 18.9, 20.3, 20.7, 21.4, 21.6, 22.0, 23.2, 23.9, 24.9, 25.2, 26.3, 26.6, 27.4, 28.6, 29.3, 30.0, 30.7, 31.2, 32.6, 34.3, 35.9, 38.5, and 39.4 degrees 2θ. In another embodiment, Compound A—Monohydrate is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 10.1, 10.7, 11.5, 13.2, 13.9, 14.3, 16.7, 17.1, 17.6, 18.3, 18.4, 18.9, 20.3, 20.7, 21.4, 21.6, 22.0, 23.2, 23.9, 24.9, 25.2, 26.3, 26.6, 27.4, 28.6, 29.3, 30.0, 30.7, 31.2, 32.6, 34.3, 35.9, 38.5, and 39.4 degrees 2θ. In another embodiment, Compound A—Monohydrate is characterized by an X-ray powder diffraction (XRPD) pattern comprising at least three diffraction angles, when measured using Cu K_{α} radiation, selected from a group consisting of about 10.1, 10.7, 11.5, 13.2, 13.9, 14.3, 16.7, 17.1, 17.6, 18.3, 18.4, 18.9, 20.3, 20.7, 21.4, 21.6, 22.0, 23.2, 23.9, 24.9, 25.2, 26.3, 26.6, 27.4, 28.6, 29.3, 30.0, 30.7, 31.2, 32.6, 34.3, 35.9, 38.5, and 39.4 degrees 2θ.

In another embodiment, Compound A—Monohydrate is characterized by an X-ray powder diffraction (XRPD) pattern comprising at least nine diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, 25.2, 26.3, 26.6, 28.6, 30.0, and 32.6 degrees 2θ. In another embodiment, Compound A—Monohydrate is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, 25.2, 26.3, 26.6, 28.6, 30.0, and 32.6 degrees 2θ. In another embodiment, Compound A—Monohydrate is characterized by an X-ray powder diffraction (XRPD) pattern comprising at least three diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, 25.2, 26.3, 26.6, 28.6, 30.0, and 32.6 degrees 2θ.

In another embodiment, Compound A—Monohydrate is characterized by an X-ray powder diffraction (XRPD) pattern comprising at least nine diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, and 26.6 degrees 2θ. In another embodiment, Compound A—Monohydrate is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, and 26.6 degrees 2θ. In another embodiment, Compound A—Monohydrate is characterized by an X-ray powder diffraction (XRPD) pattern comprising at least three diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, and 26.6 degrees 2θ.

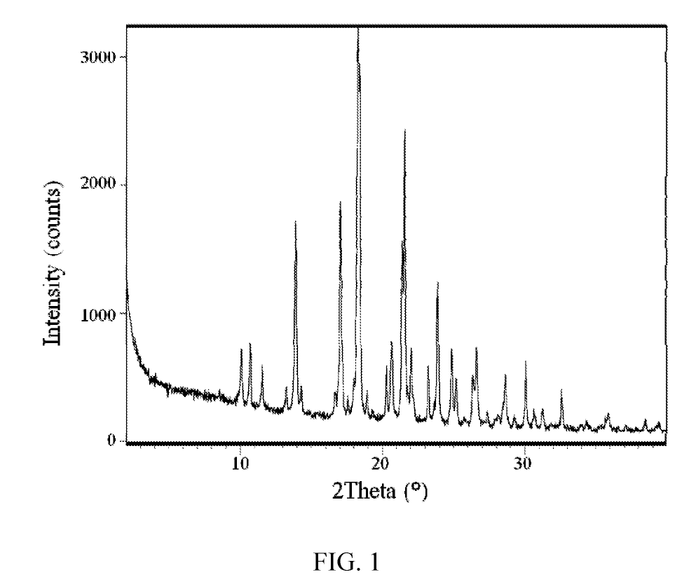

In still another embodiment, Compound A—Monohydrate is characterized by an X-ray powder diffraction (XRPD) pattern comprising diffraction angles, when measured using Cu K_{α} radiation, of about 13.9, 17.1, 18.3, 18.4, 21.4, 21.6, and 23.9 degrees 2θ. In yet another embodiment, Compound A—Monohydrate is characterized by an X-ray powder diffraction (XRPD) pattern substantially in accordance with FIG. 1.

In other embodiments, Compound A—Monohydrate is characterized by a Raman spectrum comprising at least nine peaks at positions selected from a group consisting of peaks at about 422, 450, 489, 516, 545, 575, 669, 700, 716, 733, 774, 818, 894, 918, 963, 989, 1032, 1112, 1174, 1241, 1296, 1334, 1428, 1463, 1484, 1506, 1532, 1566, 1629, 1645, 1721, 2930, 2990, and 3087 cm⁻¹. In another embodiment, Compound A—Monohydrate is characterized by a Raman spectrum comprising at least eight peaks or at least seven peaks or at least six peaks or at least five peaks or at least four three peaks at positions selected from a group consisting of peaks at about 422, 450, 489, 516, 545, 575, 669, 700, 716, 733, 774, 818, 894, 918, 963, 989, 1032, 1112, 1174, 1241, 1296, 1334, 1428, 1463, 1484, 1506, 1532, 1566, 1629, 1645, 1721, 2930, 2990, and 3087 cm⁻¹. In another embodiment, Compound A—Monohydrate is characterized by a Raman spectrum comprising at least three peaks at positions selected from a group consisting of peaks at about 422, 450, 489, 516, 545, 575, 669, 700, 716, 733, 774, 818, 894, 918, 963, 989, 1032, 1112, 1174, 1241, 1296, 1334, 1428, 1463, 1484, 1506, 1532, 1566, 1629, 1645, 1721, 2930, 2990, and 3087 cm⁻¹.

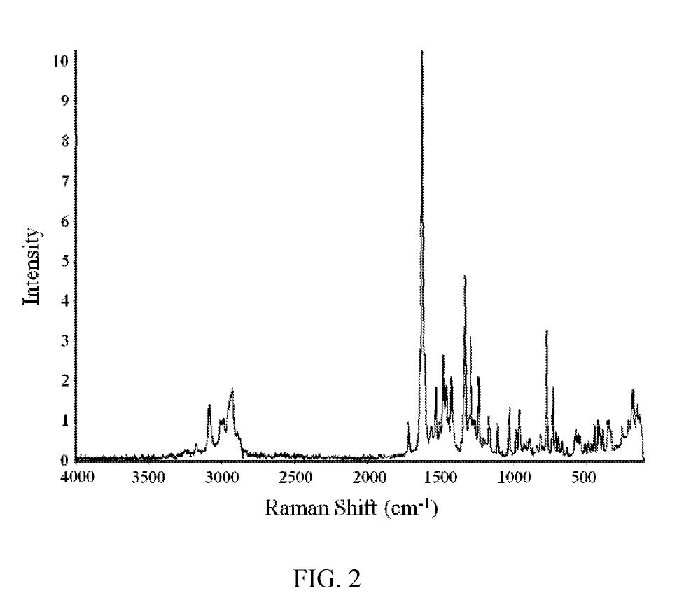

In one embodiment, Compound A—Monohydrate is characterized by a Raman spectrum comprising at least three peaks at positions selected from a group consisting of peaks at about 422, 450, 733, 774, 963, 989, 1032, 1112, 1174, 1241, 1296, 1334, 1428, 1463, 1484, 1506, 1532, 1566, 1629, 1645, 1721, 2930, 2990, and 3087 cm⁻¹. In another embodiment, Compound A—Monohydrate is characterized by a Raman spectrum comprising at least three peaks at positions selected from a group consisting of peaks at about 733, 774, 963, 1032, 1241, 1296, 1334, 1428, 1463, 1484, 1532, 1629, 1645, 2930, and 3087 cm⁻¹. In still another embodiment, Compound A—Monohydrate is characterized by a Raman spectrum comprising peaks at about 774, 1032, 1241, 1296, 1334, 1428, 1484, 1532, 1629, 2930, and 3087 cm⁻¹. In yet another embodiment, Compound A—Monohydrate is characterized by a Raman spectrum substantially in accordance with FIG. 2.

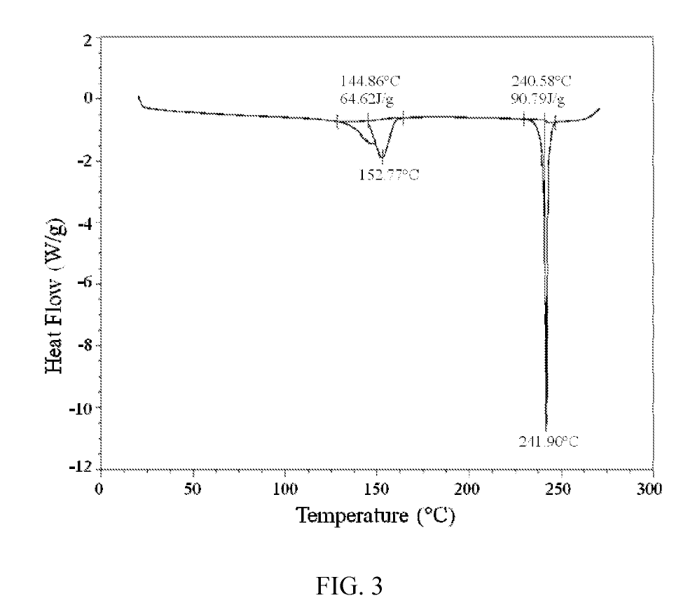

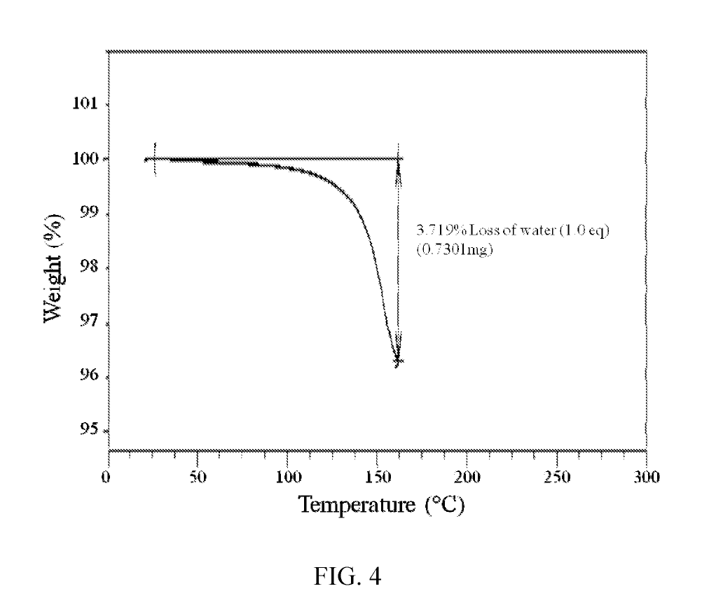

In further embodiments, Compound A—Monohydrate is characterized by a differential scanning calorimetry trace substantially in accordance with FIG. 3 and/or a thermogravimetric analysis trace substantially in accordance with FIG. 4.

In still further embodiments, as a person having ordinary skill in the art will understand, Compound A—Monohydrate is characterized by any combination of the analytical data characterizing the aforementioned embodiments. For example, in one embodiment, Compound A—Monohydrate is characterized by an X-ray powder diffraction (XRPD) pattern substantially in accordance with FIG. 1 and a Raman spectrum substantially in accordance with FIG. 2 and a differential scanning calorimetry trace substantially in accordance with FIG. 3 and a thermogravimetric analysis trace substantially in accordance with FIG. 4. In another embodiment, Compound A—Monohydrate is characterized by an X-ray powder diffraction (XRPD) pattern substantially in accordance with FIG. 1 and a Raman spectrum substantially in accordance with FIG. 2. In another embodiment, Compound A—Monohydrate is characterized by an X-ray powder diffraction (XRPD) pattern substantially in accordance with FIG. 1 and a differential scanning calorimetry trace substantially in accordance with FIG. 3. In another embodiment, Compound A—Monohydrate is characterized by an X-ray powder diffraction (XRPD) pattern substantially in accordance with FIG. 1 and a thermogravimetric analysis trace substantially in accordance with FIG. 4. In another embodiment, Compound A—Monohydrate is characterized by an X-ray powder diffraction (XRPD) pattern comprising diffraction angles, when measured using Cu K_{α} radiation, of about 13.9, 17.1, 18.3, 18.4, 21.4, 21.6, and 23.9 degrees 2θ, and a Raman spectrum comprising peaks at about 774, 1032, 1241, 1296, 1334, 1428, 1484, 1532, 1629, 2930, and 3087 cm⁻¹. In another embodiment, Compound A—Monohydrate is characterized by an X-ray powder diffraction (XRPD) pattern comprising diffraction angles, when measured using Cu K_{α} radiation, of about 13.9, 17.1, 18.3, 18.4, 21.4, 21.6, and 23.9 degrees 2θ, and a differential scanning calorimetry trace substantially in accordance with FIG. 3. In another embodiment, Compound A—Monohydrate is characterized by an X-ray powder diffraction (XRPD) pattern comprising diffraction angles, when measured using Cu K_{α} radiation, of about 13.9, 17.1, 18.3, 18.4, 21.4, 21.6, and 23.9 degrees 2θ, and a thermogravimetric analysis trace substantially in accordance with FIG. 4.

In some embodiments, a crystalline form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 1) is characterized by an X-ray powder diffraction (XRPD) pattern comprising at least nine diffraction angles, when measured using Cu K_{α} radiation, selected from a group consisting of about 4.5, 5.0, 6.0, 7.9, 9.3, 10.0, 11.2, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, 28.1, 28.7, 29.1, 30.3, 31.3, and 35.6 degrees 2θ. In another embodiment, Compound A—Non-solvated Form 1 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 4.5, 5.0, 6.0, 7.9, 9.3, 10.0, 11.2, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, 28.1, 28.7, 29.1, 30.3, 31.3, and 35.6 degrees 2θ. In another embodiment, Compound A—Non-solvated Form 1 is characterized by an X-ray powder diffraction (XRPD) pattern comprising at least three diffraction angles, when measured using Cu K_{α} radiation, selected from a group consisting of about 4.5, 5.0, 6.0, 7.9, 9.3, 10.0, 11.2, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, 28.1, 28.7, 29.1, 30.3, 31.3, and 35.6 degrees 2θ.

In another embodiment, Compound A—Non-solvated Form 1 is characterized by an X-ray powder diffraction (XRPD) pattern comprising at least nine diffraction angles, when measured using Cu K_{α} radiation, selected from a group consisting of about 4.5, 6.0, 7.9, 9.3, 10.0, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, and 28.7 degrees 2θ. In another embodiment, Compound A—Non-solvated Form 1 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 4.5, 6.0, 7.9, 9.3, 10.0, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, and 28.7 degrees 2θ. In another embodiment, Compound A—Non-solvated Form 1 is characterized by an X-ray powder diffraction (XRPD) pattern comprising at least three diffraction angles, when measured using Cu K_{α} radiation, selected from a group consisting of about 4.5, 6.0, 7.9, 9.3, 10.0, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, and 28.7 degrees 2θ.

In another embodiment, Compound A—Non-solvated Form 1 is characterized by an X-ray powder diffraction (XRPD) pattern comprising at least nine diffraction angles, when measured using Cu K_{α} radiation, selected from a group consisting of about 4.5, 9.3, 13.1, 13.3, 13.8, 15.0, 17.1, 18.2, 18.7, 19.7, 21.6, 22.6, 23.3, 23.8, 24.3, 26.0, 26.6, and 28.7 degrees 2θ. In another embodiment, Compound A—Non-solvated Form 1 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 4.5, 9.3, 13.1, 13.3, 13.8, 15.0, 17.1, 18.2, 18.7, 19.7, 21.6, 22.6, 23.3, 23.8, 24.3, 26.0, 26.6, and 28.7 degrees 2θ. In another embodiment, Compound A—Non-solvated Form 1 is characterized by an X-ray powder diffraction (XRPD) pattern comprising at least three diffraction angles, when measured using Cu K_{α} radiation, selected from a group consisting of about 4.5, 9.3, 13.1, 13.3, 13.8, 15.0, 17.1, 18.2, 18.7, 19.7, 21.6, 22.6, 23.3, 23.8, 24.3, 26.0, 26.6, and 28.7 degrees 2θ.

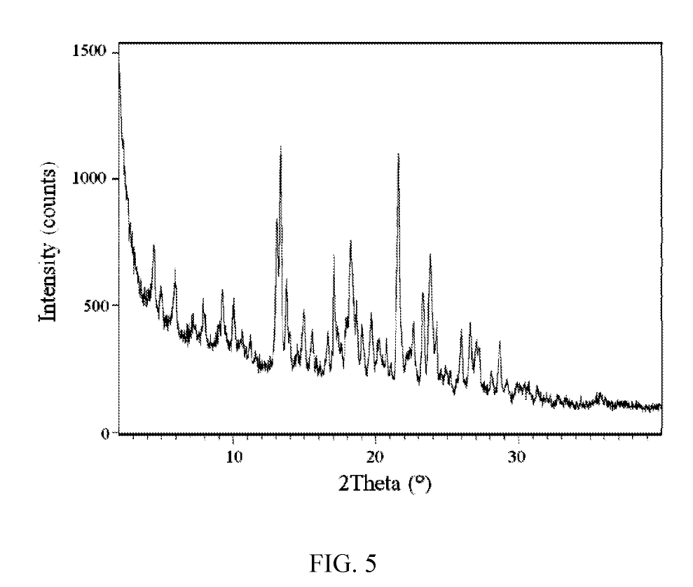

In still another embodiment, Compound A—Non-solvated Form 1 is characterized by an X-ray powder diffraction (XRPD) pattern comprising diffraction angles, when measured using Cu K_{α} radiation, of about 13.1, 13.3, 17.1, 18.2, 21.6, 23.3, and 23.8 degrees 2θ. In yet another embodiment, Compound A—Non-solvated Form 1 is characterized by an X-ray powder diffraction (XRPD) pattern substantially in accordance with FIG. 5.

In other embodiments, Compound A—Non-solvated Form 1 is characterized by a Raman spectrum comprising at least nine peaks at positions selected from a group consisting of peaks at about 450, 544, 566, 668, 726, 771, 819, 898, 978, 1035, 1110, 1176, 1242, 1273, 1329, 1424, 1470, 1484, 1511, 1534, 1626, 1681, 2930, 2999, and 3093 cm⁻¹. In another embodiment, Compound A—Non-solvated Form 1 is characterized by a Raman spectrum comprising at least eight peaks or at least seven peaks or at least six peaks or at least five peaks or at least four three peaks at positions selected from a group consisting of peaks at about 450, 544, 566, 668, 726, 771, 819, 898, 978, 1035, 1110, 1176, 1242, 1273, 1329, 1424, 1470, 1484, 1511, 1534, 1626, 1681, 2930, 2999, and 3093 cm⁻¹. In another embodiment, Compound A—Non-solvated Form 1 is characterized by a Raman spectrum comprising at least three peaks at positions selected from a group consisting of peaks at about 450, 544, 566, 668, 726, 771, 819, 898, 978, 1035, 1110, 1176, 1242, 1273, 1329, 1424, 1470, 1484, 1511, 1534, 1626, 1681, 2930, 2999, and 3093 cm⁻¹.

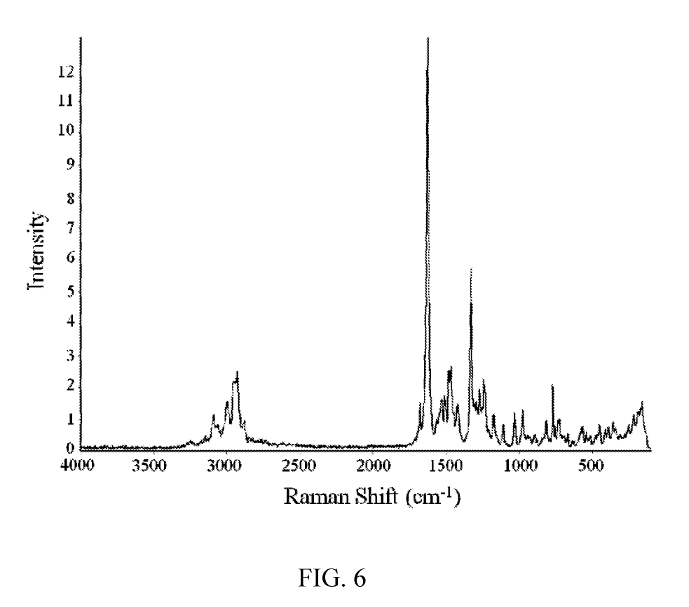

In one embodiment, Compound A—Non-solvated Form 1 is characterized by a Raman spectrum comprising at least three peaks at positions selected from a group consisting of peaks at about 726, 771, 819, 978, 1035, 1110, 1176, 1242, 1273, 1329, 1424, 1470, 1484, 1511, 1534, 1626, 1681, 2930, 2999, and 3093 cm⁻¹. In another embodiment, Compound A—Non-solvated Form 1 is characterized by a Raman spectrum comprising at least three peaks at positions selected from a group consisting of peaks at about 771, 978, 1035, 1176, 1242, 1273, 1329, 1424, 1470, 1511, 1534, 1626, 2930, and 2999 cm⁻¹. In still another embodiment, Compound A—Non-solvated Form 1 is characterized by a Raman spectrum comprising peaks at about 1242, 1329, 1470, 1626, 2930, and 2999 cm⁻¹. In yet another embodiment, Compound A—Non-solvated Form 1 is characterized by a Raman spectrum substantially in accordance with FIG. 6.

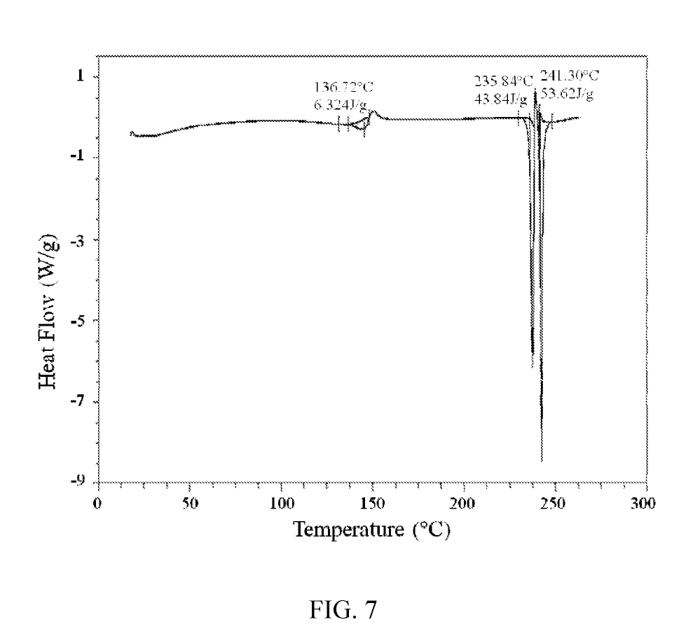

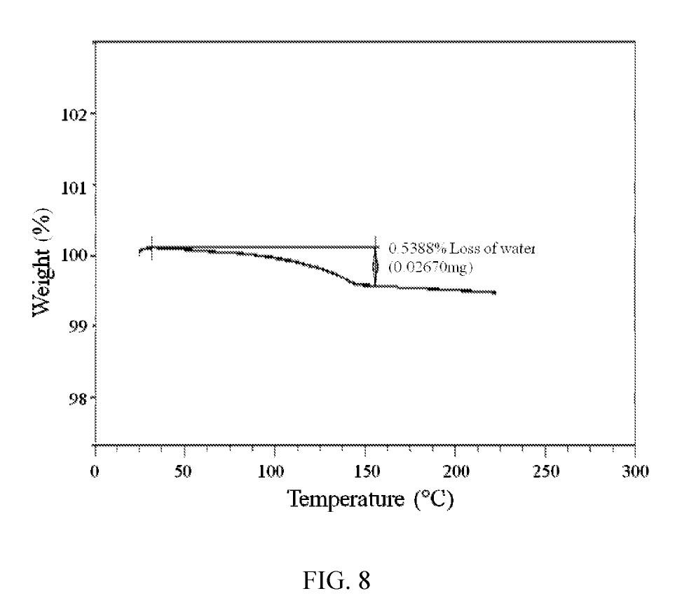

In further embodiments, Compound A—Non-solvated Form 1 is characterized by a differential scanning calorimetry trace substantially in accordance with FIG. 7 and/or a thermogravimetric analysis trace substantially in accordance with FIG. 8.

In still further embodiments, as a person having ordinary skill in the art will understand, Compound A—Non-solvated Form 1 is characterized by any combination of the analytical data characterizing the aforementioned embodiments. For example, in one embodiment, Compound A—Non-solvated Form 1 is characterized by an X-ray powder diffraction (XRPD) pattern substantially in accordance with FIG. 5 and a Raman spectrum substantially in accordance with FIG. 6 and a differential scanning calorimetry trace substantially in accordance with FIG. 7 and a thermogravimetric analysis trace substantially in accordance with FIG. 8. In another embodiment, Compound A—Non-solvated Form 1 is characterized by an X-ray powder diffraction (XRPD) pattern substantially in accordance with FIG. 5 and a Raman spectrum substantially in accordance with FIG. 6. In another embodiment, Compound A—Non-solvated Form 1 is characterized by an X-ray powder diffraction (XRPD) pattern substantially in accordance with FIG. 5 and a differential scanning calorimetry trace substantially in accordance with FIG. 7. In another embodiment, Compound A—Non-solvated Form 1 is characterized by an X-ray powder diffraction (XRPD) pattern substantially in accordance with FIG. 5 and a thermogravimetric analysis trace substantially in accordance with FIG. 8. In another embodiment, Compound A—Non-solvated Form 1 is characterized by an X-ray powder diffraction (XRPD) pattern comprising diffraction angles, when measured using Cu K_{α} radiation, of about 13.1, 13.3, 17.1, 18.2, 21.6, 23.3, and 23.8 degrees 2θ, and a Raman spectrum comprising peaks at about 1242, 1329, 1470, 1626, 2930, and 2999 cm⁻¹. In another embodiment, Compound A—Non-solvated Form 1 is characterized by an X-ray powder diffraction (XRPD) pattern comprising diffraction angles, when measured using Cu K_{α} radiation, of about 13.1, 13.3, 17.1, 18.2, 21.6, 23.3, and 23.8 degrees 2θ, and a differential scanning calorimetry trace substantially in accordance with FIG. 7. In another embodiment, Compound A—Non-solvated Form 1 is characterized by an X-ray powder diffraction (XRPD) pattern comprising diffraction angles, when measured using Cu K_{α} radiation, of about 13.1, 13.3, 17.1, 18.2, 21.6, 23.3, and 23.8 degrees 2θ, and a thermogravimetric analysis trace substantially in accordance with FIG. 8.

In some embodiments, a crystalline form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 2) is characterized by an X-ray powder diffraction (XRPD) pattern comprising at least nine diffraction angles, when measured using Cu K_{α} radiation, selected from a group consisting of about 6.4, 12.7, 14.2, 15.4, 16.1, 17.2, 17.9, 18.9, 19.6, 20.1, 21.2, 21.9, 22.8, 23.7, 24.7, 25.6, 26.6, 28.7, 29.5, 32.3, and 34.9 degrees 2θ. In another embodiment, Compound A—Non-solvated Form 2 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 6.4, 12.7, 14.2, 15.4, 16.1, 17.2, 17.9, 18.9, 19.6, 20.1, 21.2, 21.9, 22.8, 23.7, 24.7, 25.6, 26.6, 28.7, 29.5, 32.3, and 34.9 degrees 20. In another embodiment, Compound A—Non-solvated Form 2 is characterized by an X-ray powder diffraction (XRPD) pattern comprising at least three diffraction angles, when measured using Cu K_{α} radiation, selected from a group consisting of about 6.4, 12.7, 14.2, 15.4, 16.1, 17.2, 17.9, 18.9, 19.6, 20.1, 21.2, 21.9, 22.8, 23.7, 24.7, 25.6, 26.6, 28.7, 29.5, 32.3, and 34.9 degrees 2θ.

In another embodiment, Compound A—Non-solvated Form 2 is characterized by an X-ray powder diffraction (XRPD) pattern comprising at least nine diffraction angles, when measured using Cu K_{α} radiation, selected from a group consisting of about 6.4, 12.7, 14.2, 15.4, 16.1, 17.2, 17.9, 18.9, 19.6, 20.1, 21.2, 23.7, 24.7, 25.6, 26.6, and 28.7 degrees 2θ. In another embodiment, Compound A—Non-solvated Form 2 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 6.4, 12.7, 14.2, 15.4, 16.1, 17.2, 17.9, 18.9, 19.6, 20.1, 21.2, 23.7, 24.7, 25.6, 26.6, and 28.7 degrees 2θ. In another embodiment, Compound A—Non-solvated Form 2 is characterized by an X-ray powder diffraction (XRPD) pattern comprising at least three diffraction angles, when measured using Cu K_{α} radiation, selected from a group consisting of about 6.4, 12.7, 14.2, 15.4, 16.1, 17.2, 17.9, 18.9, 19.6, 20.1, 21.2, 23.7, 24.7, 25.6, 26.6, and 28.7 degrees 2θ.

In another embodiment, Compound A—Non-solvated Form 2 is characterized by an X-ray powder diffraction (XRPD) pattern comprising at least nine diffraction angles, when measured using Cu K_{α} radiation, selected from a group consisting of about 6.4, 12.7, 14.2, 15.4, 17.2, 17.9, 18.9, 20.1, 21.2, 25.6, and 26.6 degrees 2θ. In another embodiment, Compound A—Non-solvated Form 2 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 6.4, 12.7, 14.2, 15.4, 17.2, 17.9, 18.9, 20.1, 21.2, 25.6, and 26.6 degrees 2θ. In another embodiment, Compound A—Non-solvated Form 2 is characterized by an X-ray powder diffraction (XRPD) pattern comprising at least three diffraction angles, when measured using Cu K_{α} radiation, selected from a group consisting of about 6.4, 12.7, 14.2, 15.4, 17.2, 17.9, 18.9, 20.1, 21.2, 25.6, and 26.6 degrees 2θ.

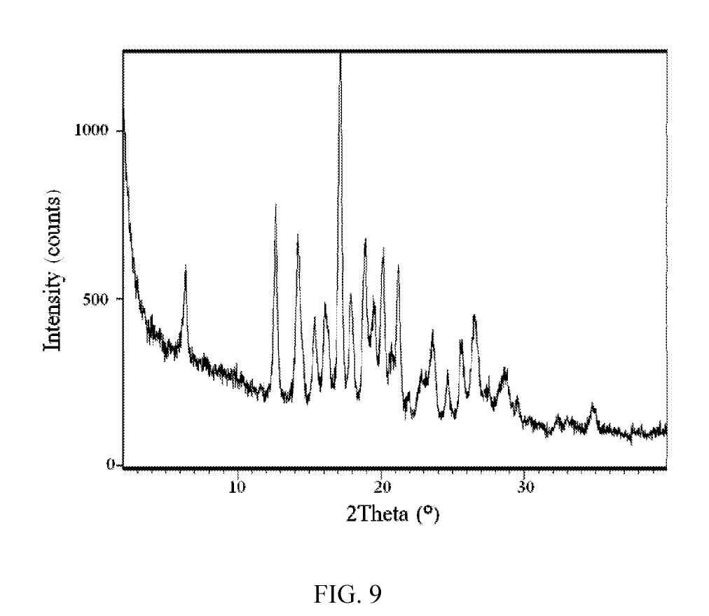

In still another embodiment, Compound A—Non-solvated Form 2 is characterized by an X-ray powder diffraction (XRPD) pattern comprising diffraction angles, when measured using Cu K_{α} radiation, of about 6.4, 12.7, 14.2, 17.2, 18.9, 20.1, and 21.2 degrees 2θ. In yet another embodiment, Compound A—Non-solvated Form 2 is characterized by an X-ray powder diffraction (XRPD) pattern substantially in accordance with FIG. 9.

In other embodiments, Compound A—Non-solvated Form 2 is characterized by a Raman spectrum comprising at least nine peaks at positions selected from a group consisting of peaks at about 417, 451, 486, 544, 576, 669, 697, 716, 730, 771, 821, 900, 964, 986, 1035, 1109, 1175, 1243, 1265, 1300, 1336, 1430, 1465, 1487, 1527, 1631, 1640, 1726, 2919, 2949, 2997, and 3082 cm⁻¹. In another embodiment, Compound A—Non-solvated Form 2 is characterized by a Raman spectrum comprising at least eight peaks or at least seven peaks or at least six peaks or at least five peaks or at least four three peaks at positions selected from a group consisting of peaks at about 417, 451, 486, 544, 576, 669, 697, 716, 730, 771, 821, 900, 964, 986, 1035, 1109, 1175, 1243, 1265, 1300, 1336, 1430, 1465, 1487, 1527, 1631, 1640, 1726, 2919, 2949, 2997, and 3082 cm⁻¹. In another embodiment, Compound A—Non-solvated Form 2 is characterized by a Raman spectrum comprising at least three peaks at positions selected from a group consisting of peaks at about 417, 451, 486, 544, 576, 669, 697, 716, 730, 771, 821, 900, 964, 986, 1035, 1109, 1175, 1243, 1265, 1300, 1336, 1430, 1465, 1487, 1527, 1631, 1640, 1726, 2919, 2949, 2997, and 3082 cm⁻¹.

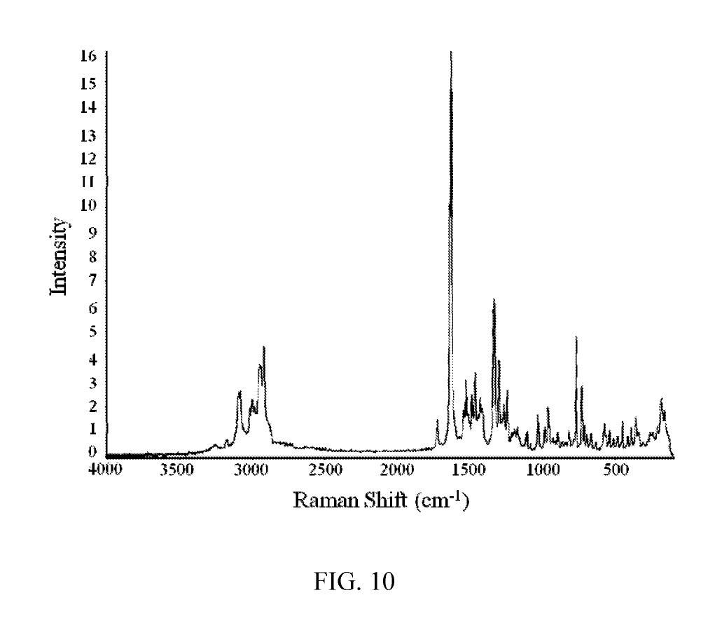

In one embodiment, Compound A—Non-solvated Form 2 is characterized by a Raman spectrum comprising at least three peaks at positions selected from a group consisting of peaks at about 451, 730, 771, 964, 1035, 1243, 1265, 1300, 1336, 1430, 1465, 1487, 1527, 1631, 1640, 1726, 2919, 2949, 2997, and 3082 cm⁻¹. In another embodiment, Compound A—Non-solvated Form 2 is characterized by a Raman spectrum comprising at least three peaks at positions selected from a group consisting of peaks at about 730, 771, 1243, 1300, 1336, 1465, 1527, 1631, 1726, 2919, and 3082 cm⁻¹. In still another embodiment, Compound A—Non-solvated Form 2 is characterized by a Raman spectrum comprising peaks at about 771, 1300, 1336, 1465, 1527, 1631, 2919, and 3082 cm⁻¹. In yet another embodiment, Compound A—Non-solvated Form 2 is characterized by a Raman spectrum substantially in accordance with FIG. 10.

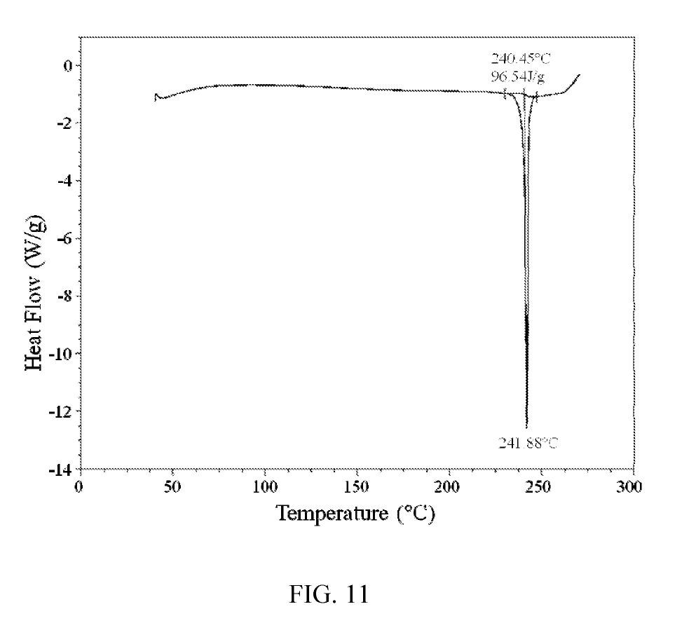

In further embodiments, Compound A—Non-solvated Form 2 is characterized by a differential scanning calorimetry trace substantially in accordance with FIG. 11.

In still further embodiments, as a person having ordinary skill in the art will understand, Compound A—Non-solvated Form 2 is characterized by any combination of the analytical data characterizing the aforementioned embodiments. For example, in one embodiment, Compound A—Non-solvated Form 2 is characterized by an X-ray powder diffraction (XRPD) pattern substantially in accordance with FIG. 9 and a Raman spectrum substantially in accordance with FIG. 10 and a differential scanning calorimetry trace substantially in accordance with FIG. 11. In another embodiment, Compound A—Non-solvated Form 2 is characterized by an X-ray powder diffraction (XRPD) pattern substantially in accordance with FIG. 9 and a Raman spectrum substantially in accordance with FIG. 10. In another embodiment, Compound A—Non-solvated Form 2 is characterized by an X-ray powder diffraction (XRPD) pattern substantially in accordance with FIG. 9 and a differential scanning calorimetry trace substantially in accordance with FIG. 11. In another embodiment, Compound A—Non-solvated Form 2 is characterized by an X-ray powder diffraction (XRPD) pattern comprising diffraction angles, when measured using Cu K_{α} radiation, of about 6.4, 12.7, 14.2, 17.2, 18.9, 20.1, and 21.2 degrees 2θ, and a Raman spectrum comprising peaks at about 771, 1300, 1336, 1465, 1527, 1631, 2919, and 3082 cm⁻¹. In another embodiment, Compound A—Non-solvated Form 2 is characterized by an X-ray powder diffraction (XRPD) pattern comprising diffraction angles, when measured using Cu K_{α} radiation, of about 6.4, 12.7, 14.2, 17.2, 18.9, 20.1, and 21.2 degrees 2θ, and a differential scanning calorimetry trace substantially in accordance with FIG. 11.

In some embodiments, a crystalline form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 3) is characterized by an X-ray powder diffraction (XRPD) pattern comprising at least nine diffraction angles, when measured using Cu K_{α} radiation, selected from a group consisting of about 9.6, 11.0, 11.7, 13.8, 14.3, 15.3, 16.6, 17.2, 17.5, 18.8, 19.3, 20.3, 21.1, 21.4, 22.0, 23.0, 23.6, 24.5, 25.8, 26.2, 27.4, 27.7, 28.6, 29.6, 30.8, 31.0, 31.4, 32.3, 33.3, 35.9, and 39.2 degrees 2θ. In another embodiment, Compound A—Non-solvated Form 3 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 9.6, 11.0, 11.7, 13.8, 14.3, 15.3, 16.6, 17.2, 17.5, 18.8, 19.3, 20.3, 21.1, 21.4, 22.0, 23.0, 23.6, 24.5, 25.8, 26.2, 27.4, 27.7, 28.6, 29.6, 30.8, 31.0, 31.4, 32.3, 33.3, 35.9, and 39.2 degrees 2θ. In another embodiment, Compound A—Non-solvated Form 3 is characterized by an X-ray powder diffraction (XRPD) pattern comprising at least three diffraction angles, when measured using Cu K_{α} radiation, selected from a group consisting of about 9.6, 11.0, 11.7, 13.8, 14.3, 15.3, 16.6, 17.2, 17.5, 18.8, 19.3, 20.3, 21.1, 21.4, 22.0, 23.0, 23.6, 24.5, 25.8, 26.2, 27.4, 27.7, 28.6, 29.6, 30.8, 31.0, 31.4, 32.3, 33.3, 35.9, and 39.2 degrees 2θ.

In another embodiment, Compound A—Non-solvated Form 3 is characterized by an X-ray powder diffraction (XRPD) pattern comprising at least nine diffraction angles, when measured using Cu K_{α} radiation, selected from a group consisting of about 9.6, 11.0, 13.8, 14.3, 15.3, 16.6, 17.5, 18.8, 19.3, 20.3, 21.1, 21.4, 22.0, 24.5, 26.2, 27.4, 27.7, 28.6, 29.6, 31.0, 31.4, 32.3, and 33.3 degrees 2θ. In another embodiment, Compound A—Non-solvated Form 3 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 9.6, 11.0, 13.8, 14.3, 15.3, 16.6, 17.5, 18.8, 19.3, 20.3, 21.1, 21.4, 22.0, 24.5, 26.2, 27.4, 27.7, 28.6, 29.6, 31.0, 31.4, 32.3, and 33.3 degrees 2θ. In another embodiment, Compound A—Non-solvated Form 3 is characterized by an X-ray powder diffraction (XRPD) pattern comprising at least three diffraction angles, when measured using Cu K_{α} radiation, selected from a group consisting of about 9.6, 11.0, 13.8, 14.3, 15.3, 16.6, 17.5, 18.8, 19.3, 20.3, 21.1, 21.4, 22.0, 24.5, 26.2, 27.4, 27.7, 28.6, 29.6, 31.0, 31.4, 32.3, and 33.3 degrees 2θ.

In another embodiment, Compound A—Non-solvated Form 3 is characterized by an X-ray powder diffraction (XRPD) pattern comprising at least nine diffraction angles, when measured using Cu K_{α} radiation, selected from a group consisting of about 9.6, 11.0, 13.8, 15.3, 17.5, 20.3, 21.4, 22.0, 24.5, 26.2, and 27.4 degrees 2θ. In another embodiment, Compound A—Non-solvated Form 3 is characterized by an X-ray powder diffraction (XRPD) pattern comprising at least eight diffraction angles or at least seven diffraction angles or at least six diffraction angles or at least five diffraction angles or at least four diffraction angles, when measured using Cu K_{α} radiation, selected from a group consisting of about 9.6, 11.0, 13.8, 15.3, 17.5, 20.3, 21.4, 22.0, 24.5, 26.2, and 27.4 degrees 2θ. In another embodiment, Compound A—Non-solvated Form 3 is characterized by an X-ray powder diffraction (XRPD) pattern comprising at least three diffraction angles, when measured using Cu K_{α} radiation, selected from a group consisting of about 9.6, 11.0, 13.8, 15.3, 17.5, 20.3, 21.4, 22.0, 24.5, 26.2, and 27.4 degrees 2θ.

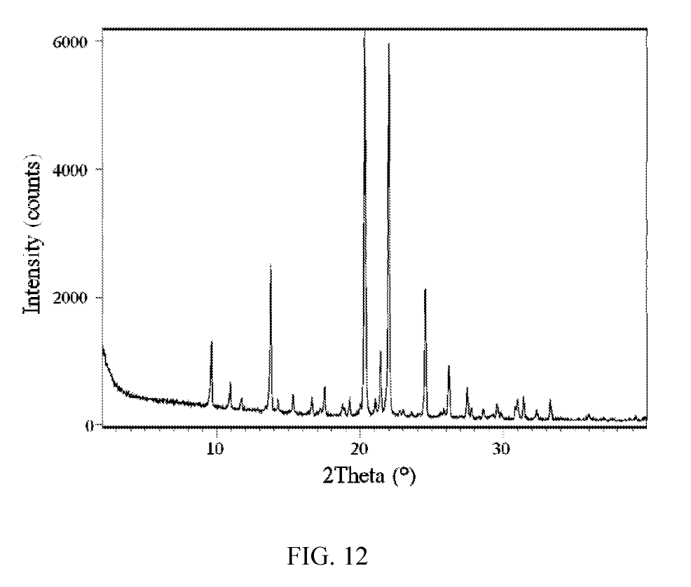

In still another embodiment, Compound A—Non-solvated Form 3 is characterized by an X-ray powder diffraction (XRPD) pattern comprising diffraction angles, when measured using Cu K_{α} radiation, of about 9.6, 13.8, 20.3, 21.4, 22.0, 24.5, and 26.2 degrees 2θ. In yet another embodiment, Compound A—Non-solvated Form 3 is characterized by an X-ray powder diffraction (XRPD) pattern substantially in accordance with FIG. 12.

In other embodiments, Compound A—Non-solvated Form 3 is characterized by a Raman spectrum comprising at least nine peaks at positions selected from a group consisting of peaks at about 454, 493, 572, 639, 728, 769, 819, 841, 923, 978, 1037, 1109, 1190, 1239, 1287, 1331, 1429, 1464, 1485, 1509, 1542, 1631, 1714, 2951, 2994, 3078, and 3093 cm⁻¹. In another embodiment, Compound A—Non-solvated Form 3 is characterized by a Raman spectrum comprising at least eight peaks or at least seven peaks or at least six peaks or at least five peaks or at least four three peaks at positions selected from a group consisting of peaks at about 454, 493, 572, 639, 728, 769, 819, 841, 923, 978, 1037, 1109, 1190, 1239, 1287, 1331, 1429, 1464, 1485, 1509, 1542, 1631, 1714, 2951, 2994, 3078, and 3093 cm⁻¹. In another embodiment, Compound A—Non-solvated Form 3 is characterized by a Raman spectrum comprising at least three peaks at positions selected from a group consisting of peaks at about 454, 493, 572, 639, 728, 769, 819, 841, 923, 978, 1037, 1109, 1190, 1239, 1287, 1331, 1429, 1464, 1485, 1509, 1542, 1631, 1714, 2951, 2994, 3078, and 3093 cm⁻¹.

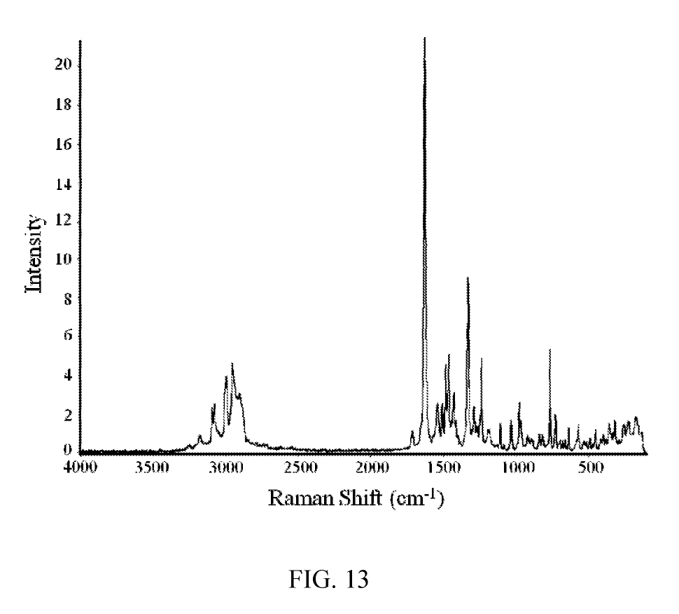

In one embodiment, Compound A—Non-solvated Form 3 is characterized by a Raman spectrum comprising at least three peaks at positions selected from a group consisting of peaks at about 572, 728, 769, 978, 1037, 1109, 1239, 1287, 1331, 1429, 1464, 1485, 1509, 1542, 1631, 1714, 2951, 2994, 3078, and 3093 cm⁻¹. In another embodiment, Compound A—Non-solvated Form 3 is characterized by a Raman spectrum comprising at least three peaks at positions selected from a group consisting of peaks at about 769, 978, 1239, 1331, 1429, 1464, 1485, 1509, 1542, 1631, 2951, and 2994 cm⁻¹. In still another embodiment, Compound A—Non-solvated Form 3 is characterized by a Raman spectrum comprising peaks at about 769, 1239, 1331, 1464, 1485, 1631, 2951, and 2994 cm⁻¹. In yet another embodiment, Compound A—Non-solvated Form 3 is characterized by a Raman spectrum substantially in accordance with FIG. 13.

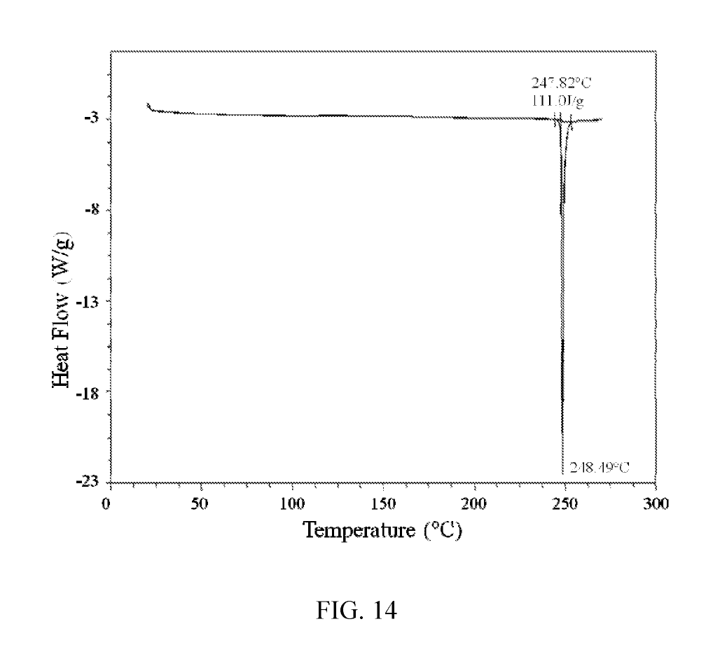

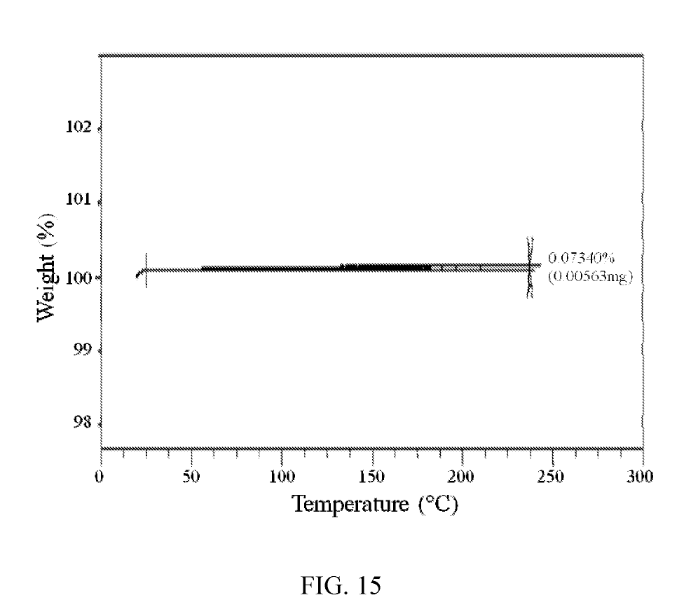

In further embodiments, Compound A—Non-solvated Form 3 is characterized by a differential scanning calorimetry trace substantially in accordance with FIG. 14 and/or a thermogravimetric analysis trace substantially in accordance with FIG. 15.

In still further embodiments, as a person having ordinary skill in the art will understand, Compound A—Non-solvated Form 3 is characterized by any combination of the analytical data characterizing the aforementioned embodiments. For example, in one embodiment, Compound A—Non-solvated Form 3 is characterized by an X-ray powder diffraction (XRPD) pattern substantially in accordance with FIG. 12 and a Raman spectrum substantially in accordance with FIG. 13 and a differential scanning calorimetry trace substantially in accordance with FIG. 14 and a thermogravimetric analysis trace substantially in accordance with FIG. 15. In another embodiment, Compound A—Non-solvated Form 3 is characterized by an X-ray powder diffraction (XRPD) pattern substantially in accordance with FIG. 12 and a Raman spectrum substantially in accordance with FIG. 13. In another embodiment, Compound A—Non-solvated Form 3 is characterized by an X-ray powder diffraction (XRPD) pattern substantially in accordance with FIG. 12 and a differential scanning calorimetry trace substantially in accordance with FIG. 14. In another embodiment, Compound A—Non-solvated Form 3 is characterized by an X-ray powder diffraction (XRPD) pattern substantially in accordance with FIG. 12 and a thermogravimetric analysis trace substantially in accordance with FIG. 15. In another embodiment, Compound A—Non-solvated Form 3 is characterized by an X-ray powder diffraction (XRPD) pattern comprising diffraction angles, when measured using Cu K_{α} radiation, of about 9.6, 13.8, 20.3, 21.4, 22.0, 24.5, and 26.2 degrees 2θ, and a Raman spectrum comprising peaks at about 769, 1239, 1331, 1464, 1485, 1631, 2951, and 2994 cm⁻¹. In another embodiment, Compound A—Non-solvated Form 3 is characterized by an X-ray powder diffraction (XRPD) pattern comprising diffraction angles, when measured using Cu K_{α} radiation, of about 9.6, 13.8, 20.3, 21.4, 22.0, 24.5, and 26.2 degrees 2θ, and a differential scanning calorimetry trace substantially in accordance with FIG. 14. In another embodiment, Compound A—Non-solvated Form 3 is characterized by an X-ray powder diffraction (XRPD) pattern comprising diffraction angles, when measured using Cu K_{α} radiation, of about 9.6, 13.8, 20.3, 21.4, 22.0, 24.5, and 26.2 degrees 2θ, and a thermogravimetric analysis trace substantially in accordance with FIG. 15.

An XRPD pattern will be understood to comprise a diffraction angle (expressed in degrees 2θ) of “about” a value specified herein when the XRPD pattern comprises a diffraction angle within ±0.3 degrees 2θ of the specified value. Further, it is well known and understood to those skilled in the art that the apparatus employed, humidity, temperature, orientation of the powder crystals, and other parameters involved in obtaining an X-ray powder diffraction (XRPD) pattern may cause some variability in the appearance, intensities, and positions of the lines in the diffraction pattern. An X-ray powder diffraction pattern that is “substantially in accordance” with that of FIG. 1, 5, 9, or 12 provided herein is an XRPD pattern that would be considered by one skilled in the art to represent a compound possessing the same crystal form as the compound that provided the XRPD pattern of FIG. 1, 5, 9, or 12. That is, the XRPD pattern may be identical to that of FIG. 1, 5, 9, or 12, or more likely it may be somewhat different. Such an XRPD pattern may not necessarily show each of the lines of any one of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide, with FIG. 1 and, using expertise and knowledge in the art, readily determine whether the XRPD pattern of the sample is substantially in accordance with the XRPD pattern of Compound A—Monohydrate. If the XRPD pattern is substantially in accordance with FIG. 1, the sample form can be readily and accurately identified as having the same form as Compound A—Monohydrate. Similarly, if an XRPD pattern of a sample of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide is substantially in accordance with FIG. 5, the sample form can be readily and accurately identified as having the same form as Compound A—Non-solvated Form 1. Similarly, if an XRPD pattern of a sample of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide is substantially in accordance with FIG. 9, the sample form can be readily and accurately identified as having the same form as Compound A—Non-solvated Form 2. Similarly, if an XRPD pattern of a sample of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide is substantially in accordance with FIG. 12, the sample form can be readily and accurately identified as having the same form as Compound A—Non-solvated Form 3.

A Raman spectrum will be understood to comprise a peak (expressed in cm⁻¹) of “about” a value specified herein when the Raman spectrum comprises a peak within ±5.0 cm⁻¹ of the specified value. Further, it is also well known and understood to those skilled in the art that the apparatus employed, humidity, temperature, orientation of the powder crystals, and other parameters involved in obtaining a Raman spectrum may cause some variability in the appearance, intensities, and positions of the peaks in the spectrum. A Raman spectrum that is “substantially in accordance” with that of FIG. 2, 6, 10, or 13 provided herein is a Raman spectrum that would be considered by one skilled in the art to represent a compound possessing the same crystal form as the compound that provided the Raman spectrum of FIG. 2, 6, 10, or 13. That is, the Raman spectrum may be identical to that of FIG. 2, 6, 10, or 13, or more likely it may be somewhat different. Such a Raman spectrum may not necessarily show each of the peaks of any one of the spectra presented herein, and/or may show a slight change in appearance, intensity, or a shift in position of said peaks resulting from differences in the conditions involved in obtaining the data. A person skilled in the art is capable of determining if a sample of a crystalline compound has the same form as, or a different form from, a form disclosed herein by comparison of their Raman spectra. For example, one skilled in the art can overlay a Raman spectrum of a sample of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide, with FIG. 2 and, using expertise and knowledge in the art, readily determine whether the Raman spectrum of the sample is substantially in accordance with the Raman spectrum of Compound A—Monohydrate. If the Raman spectrum is substantially in accordance with FIG. 6, the sample form can be readily and accurately identified as having the same form as Compound A—Non-solvated Form 1. Similarly, if the Raman spectrum is substantially in accordance with FIG. 10, the sample form can be readily and accurately identified as having the same form as Compound A—Non-solvated Form 2. Similarly, if the Raman spectrum is substantially in accordance with FIG. 13, the sample form can be readily and accurately identified as having the same form as Compound A—Non-solvated Form 3.

“Compound of the invention” means 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide, and in some embodiments, specifically the crystalline form defined herein as Compound A—Monohydrate, or in some embodiments, specifically the crystalline form defined herein as Compound A—Non-solvated Form 1, or in some embodiments, specifically the crystalline form defined herein as Compound A—Non-solvated Form 2, or in some embodiments, specifically the crystalline form defined herein as Compound A—Non-solvated Form 3.

The invention includes a therapeutic method for treating or ameliorating a RET-mediated disorder in a human in need thereof comprising administering to a human in need thereof an effective amount of a compound of the invention or a composition comprising an effective amount of a compound of the invention and an optional pharmaceutically acceptable carrier. In certain embodiments, the RET-mediated disorder is irritable bowel syndrome (IBS) including diarrhea-predominant, constipation-predominant or alternating stool pattern, functional bloating, functional constipation, functional diarrhea, unspecified functional bowel disorder, functional abdominal pain syndrome, chronic idiopathic constipation, functional esophageal disorders, functional gastroduodenal disorders, functional anorectal pain, inflammatory bowel disease, proliferative diseases such as non-small cell lung cancer, hepatocellular carcinoma, colorectal cancer, medullary thyroid cancer, follicular thyroid cancer, anaplastic thyroid cancer, papillary thyroid cancer, brain tumors, peritoneal cavity cancer, solid tumors, other lung cancer, head and neck cancer, gliomas, neuroblastomas, Von Hippel-Lindau Syndrome and kidney tumors, breast cancer, fallopian tube cancer, ovarian cancer, transitional cell cancer, prostate cancer, caner of the esophagus and gastroesophageal junction, biliary cancer and adenocarcinoma. In certain embodiments, compounds described herein are useful for treating irritable bowel syndrome. In certain embodiments, compounds described herein are useful for treating cancer.

In another aspect, this invention relates to a compound of the invention for use in the treatment of irritable bowel syndrome (IBS) including diarrhea-predominant, constipation-predominant or alternating stool pattern, functional bloating, functional constipation, functional diarrhea, unspecified functional bowel disorder, functional abdominal pain syndrome, chronic idiopathic constipation, functional esophageal disorders, functional gastroduodenal disorders, functional anorectal pain, inflammatory bowel disease, non-small cell lung cancer, hepatocellular carcinoma, colorectal cancer, medullary thyroid cancer, follicular thyroid cancer, anaplastic thyroid cancer, papillary thyroid cancer, brain tumors, peritoneal cavity cancer, solid tumors, other lung cancer, head and neck cancer, gliomas, neuroblastomas, Von Hippel-Lindau Syndrome and kidney tumors, breast cancer, fallopian tube cancer, ovarian cancer, transitional cell cancer, prostate cancer, cancer of the esophagus and gastroesophageal junction, biliary cancer and adenocarcinoma.

In another aspect, the invention includes the use of a compound of the invention in therapy, in particular, for use in therapy wherein the subject is a human. The invention further includes the use of a compound of the invention as an active therapeutic substance, in particular in the treatment of RET-mediated disorders. In particular, the invention includes the use of a compound of the invention in the treatment of irritable bowel syndrome (IBS) including diarrhea-predominant, constipation-predominant or alternating stool pattern, functional bloating, functional constipation, functional diarrhea, unspecified functional bowel disorder, functional abdominal pain syndrome, chronic idiopathic constipation, functional esophageal disorders, functional gastroduodenal disorders, functional anorectal pain, inflammatory bowel disease, non-small cell lung cancer, hepatocellular carcinoma, colorectal cancer, medullary thyroid cancer, follicular thyroid cancer, anaplastic thyroid cancer, papillary thyroid cancer, brain tumors, peritoneal cavity cancer, solid tumors, other lung cancer, head and neck cancer, gliomas, neuroblastomas, Von Hippel-Lindau Syndrome and kidney tumors, breast cancer, fallopian tube cancer, ovarian cancer, transitional cell cancer, prostate cancer, cancer of the esophagus and gastroesophageal junction, biliary cancer and adenocarcinoma. In another aspect, the invention includes the use of a compound of the invention in the treatment of irritable bowel syndrome. In another aspect, the invention includes the use of a compound of the invention in the treatment of cancer.

In another aspect, the invention includes the use of a compound of the invention in the manufacture of a medicament for use in the treatment of the above disorders. In another aspect, the invention includes the use of a compound of the invention in the manufacture of a medicament for use in the treatment of irritable bowel syndrome. In another aspect, the invention includes the use of a compound of the invention in the manufacture of a medicament for use in the treatment of cancer.

As used herein, the term “RET-mediated disorder” means any disease, disorder, or other pathological condition in which Rearranged during Transfection (RET) kinase is known to play a role. Accordingly, in some embodiments, the present disclosure relates to treating or lessening the severity of one or more diseases in which RET is known to play a role.

As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician. The effective amount of a compound of the invention in such a therapeutic method is about 0.1 to 100 mg per kg patient body weight per day which can be administered in single or multiple doses. In some embodiments, the dosage level will be about 0.1 to about 25 mg/kg per day. In some embodiments, the dosage level will be about 0.1 to about 10 mg/kg per day. A suitable dosage level may be about 0.1 to 25 mg/kg per day, about 0.1 to 10 mg/kg per day, or about 0.1 to 5 mg/kg per day. Within this range the dosage may be 0.1 to 0.5, 0.5 to 1.0, 1.0 to 5.0, 5.0 to 10.0, or 10 to 25 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compound may be administered on a regimen of 1 to 4 times per day, preferably once or twice per day. In some embodiments, a compound described herein is administered one or more times per day, for multiple days. In some embodiments, the dosing regimen is continued for days, weeks, months, or years.

It is to be understood, however, that the specific dose level and frequency of dosage for any particular patient may be varied and will depend upon a variety of factors including age, body weight, hereditary characteristics, general health, gender, diet, mode and time of administration, rate of excretion, drug combination, and the nature and severity of the particular condition being treated.

Administration methods include administering an effective amount of a compound or composition of the invention at different times during the course of therapy or concurrently in a combination form. The methods of the invention include all known therapeutic treatment regimens.

The compounds and compositions of the present invention can be combined with other compounds and compositions having related utilities to prevent and treat the condition or disease of interest, such as a proliferative disorder. Selection of the appropriate agents for use in combination therapies can be made by one of ordinary skill in the art. The combination of therapeutic agents may act synergistically to effect the treatment or prevention of the various disorders. Using this approach, one may be able to achieve therapeutic efficacy with lower dosages of each agent, thus reducing the potential for adverse side effects. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

Combination therapy includes co-administration of the compound of the invention and said other agent, sequential administration of the compound of the invention and the other agent, administration of a composition containing the compound of the invention and the other agent, or simultaneous administration of separate compositions containing the compound of the invention and the other agent.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The present invention is also directed to a pharmaceutical composition comprising a compound of the invention and a pharmaceutically acceptable carrier. The present invention is further directed to a method of preparing a pharmaceutical composition comprising admixing a compound of the invention and a pharmaceutically acceptable carrier.

“Pharmaceutically acceptable carrier” means any one or more compounds and/or compositions that are of sufficient purity and quality for use in the formulation of the compound of the invention that, when appropriately administered to a human, do not produce an adverse reaction, and that are used as a vehicle for a drug substance (i.e. a compound of the present invention). Carriers may include excipients, diluents, granulating and/or dispersing agents, surface active agents and/or emulsifiers, binding agents, preservatives, buffering agents, lubricating agents, and natural oils.

The invention further includes the process for making the composition comprising mixing a compound of the invention and an optional pharmaceutically acceptable carrier; and includes those compositions resulting from such a process, which process includes conventional pharmaceutical techniques. For example, a compound of the invention may be nanomilled prior to formulation. A compound of the invention may also be prepared by grinding, micronizing or other particle size reduction methods known in the art. Such methods include, but are not limited to, those described in U.S. Pat. Nos. 4,826,689, 5,145,684, 5,298,262, 5,302,401, 5,336,507, 5,340,564, 5,346,702, 5,352,459, 5,354,560, 5,384,124, 5,429,824, 5,503,723, 5,510,118, 5,518,187, 5,518,738, 5,534,270, 5,536,508, 5,552,160, 5,560,931, 5,560,932, 5,565,188, 5,569,448, 5,571,536, 5,573,783, 5,580,579, 5,585,108, 5,587,143, 5,591,456, 5,622,938, 5,662,883, 5,665,331, 5,718,919, 5,747,001, PCT applications WO 93/25190, WO 96/24336, and WO 98/35666, each of which is incorporated herein by reference. The pharmaceutical compositions of the invention may be prepared using techniques and methods known to those skilled in the art. Some of the methods commonly used in the art are described in Remington's Pharmaceutical Sciences (Mack Publishing Company), the entire teachings of which are incorporated herein by reference.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders.

The oral composition is preferably formulated as a homogeneous composition, wherein the drug substance (i.e. a compound of the present invention) is dispersed evenly throughout the mixture, which may be readily subdivided into dosage units containing equal amounts of the compound of the invention. Preferably, the compositions are prepared by mixing a compound of the invention with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myri state, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

A compound of the invention may also be administered via a delayed release composition, wherein the composition includes a compound of the invention and a biodegradable slow release carrier (e.g. a polymeric carrier) or a pharmaceutically acceptable non-biodegradable slow release carrier (e.g. an ion exchange carrier).

Biodegradable and non-biodegradable delayed release carriers are well known in the art. Biodegradable carriers are used to form particles or matrices which retain a drug substance(s) (i.e. a compound of the present invention) and which slowly degrade/dissolve in a suitable environment (e.g. aqueous, acidic, basic and the like) to release the drug substance(s). Such particles degrade/dissolve in body fluids to release the drug substance(s) (i.e. compounds of the present invention) therein. The particles are preferably nanoparticles (e.g. in the range of about 1 to 500 nm in diameter, preferably about 50-200 nm in diameter, and most preferably about 100 nm in diameter). In a process for preparing a slow release composition, a slow release carrier and the compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

In certain embodiments, this invention relates to a pharmaceutical composition comprising Compound A. In another embodiment, this invention relates to a pharmaceutical composition comprising Compound A wherein at least 10% by weight of Compound A is present as Compound A—Monohydrate. In another embodiment, this invention relates to a pharmaceutical composition comprising Compound A wherein at least 20% by weight, or at least 30% by weight, or at least 40% by weight, or at least 50% by weight, or at least 60% by weight, or at least 70% by weight, or at least 80% by weight, or at least 90% by weight of Compound A is present as Compound A—Monohydrate. In another embodiment, this invention relates to a pharmaceutical composition comprising Compound A wherein at least 95% by weight, or at least 96% by weight, or at least 97% by weight, or at least 98% by weight, or at least 99% by weight, or at least 99.5% by weight, or at least 99.8% by weight, or at least 99.9% by weight of Compound A is present as Compound A—Monohydrate.

In another embodiment, this invention relates to a pharmaceutical composition comprising Compound A wherein at least 10% by weight of Compound A is present as Compound A—Non-solvated Form 1. In another embodiment, this invention relates to a pharmaceutical composition comprising Compound A wherein at least 20% by weight, or at least 30% by weight, or at least 40% by weight, or at least 50% by weight, or at least 60% by weight, or at least 70% by weight, or at least 80% by weight, or at least 90% by weight of Compound A is present as Compound A—Non-solvated Form 1. In another embodiment, this invention relates to a pharmaceutical composition comprising Compound A wherein at least 95% by weight, or at least 96% by weight, or at least 97% by weight, or at least 98% by weight, or at least 99% by weight, or at least 99.5% by weight, or at least 99.8% by weight, or at least 99.9% by weight of Compound A is present as Compound A—Non-solvated Form 1.

In another embodiment, this invention relates to a pharmaceutical composition comprising Compound A wherein at least 10% by weight of Compound A is present as Compound A—Non-solvated Form 2. In another embodiment, this invention relates to a pharmaceutical composition comprising Compound A wherein at least 20% by weight, or at least 30% by weight, or at least 40% by weight, or at least 50% by weight, or at least 60% by weight, or at least 70% by weight, or at least 80% by weight, or at least 90% by weight of Compound A is present as Compound A—Non-solvated Form 2. In another embodiment, this invention relates to a pharmaceutical composition comprising Compound A wherein at least 95% by weight, or at least 96% by weight, or at least 97% by weight, or at least 98% by weight, or at least 99% by weight, or at least 99.5% by weight, or at least 99.8% by weight, or at least 99.9% by weight of Compound A is present as Compound A—Non-solvated Form 2.

In another embodiment, this invention relates to a pharmaceutical composition comprising Compound A wherein at least 10% by weight of Compound A is present as Compound A—Non-solvated Form 3. In another embodiment, this invention relates to a pharmaceutical composition comprising Compound A wherein at least 20% by weight, or at least 30% by weight, or at least 40% by weight, or at least 50% by weight, or at least 60% by weight, or at least 70% by weight, or at least 80% by weight, or at least 90% by weight of Compound A is present as Compound A—Non-solvated Form 3. In another embodiment, this invention relates to a pharmaceutical composition comprising Compound A wherein at least 95% by weight, or at least 96% by weight, or at least 97% by weight, or at least 98% by weight, or at least 99% by weight, or at least 99.5% by weight, or at least 99.8% by weight, or at least 99.9% by weight of Compound A is present as Compound A—Non-solvated Form 3.

In another embodiment, this invention relates to a pharmaceutical composition comprising Compound A wherein not more than 90% by weight of Compound A is amorphous. In another embodiment, this invention relates to a pharmaceutical composition comprising Compound A wherein not more than 80% by weight, or not more than 70% by weight, or not more than 60% by weight, or not more than 50% by weight, or not more than 40% by weight, or not more than 30% by weight, or not more than 20% by weight, or not more than 10% by weight of Compound A is amorphous. In another embodiment, this invention relates to a pharmaceutical composition comprising Compound A wherein not more than 5% by weight, or not more than 4% by weight, or not more than 3% by weight, or not more than 2% by weight, or not more than 1% by weight, or not more than 0.5% by weight, or not more than 0.2% by weight, or not more than 0.1% by weight of Compound A is amorphous.

In another embodiment, this invention relates to a pharmaceutical composition comprising Compound A wherein not more than 90% by weight of Compound A is present in a form other than Compound A—Monohydrate. In another embodiment, this invention relates to a pharmaceutical composition comprising Compound A wherein not more than 80% by weight, or not more than 70% by weight, or not more than 60% by weight, or not more than 50% by weight, or not more than 40% by weight, or not more than 30% by weight, or not more than 20% by weight, or not more than 10% by weight of Compound A is present in a form other than Compound A—Monohydrate. In another embodiment, this invention relates to a pharmaceutical composition comprising Compound A wherein not more than 5% by weight, or not more than 4% by weight, or not more than 3% by weight, or not more than 2% by weight, or not more than 1% by weight, or not more than 0.5% by weight, or not more than 0.2% by weight, or not more than 0.1% by weight of Compound A is present in a form other than Compound A—Monohydrate.

In another embodiment, this invention relates to a pharmaceutical composition comprising Compound A wherein not more than 90% by weight of Compound A is present in a form other than Compound A—Non-solvated Form 1. In another embodiment, this invention relates to a pharmaceutical composition comprising Compound A wherein not more than 80% by weight, or not more than 70% by weight, or not more than 60% by weight, or not more than 50% by weight, or not more than 40% by weight, or not more than 30% by weight, or not more than 20% by weight, or not more than 10% by weight of Compound A is present in a form other than Compound A—Non-solvated Form 1. In another embodiment, this invention relates to a pharmaceutical composition comprising Compound A wherein not more than 5% by weight, or not more than 4% by weight, or not more than 3% by weight, or not more than 2% by weight, or not more than 1% by weight, or not more than 0.5% by weight, or not more than 0.2% by weight, or not more than 0.1% by weight of Compound A is present in a form other than Compound A—Non-solvated Form 1.

In another embodiment, this invention relates to a pharmaceutical composition comprising Compound A wherein not more than 90% by weight of Compound A is present in a form other than Compound A—Non-solvated Form 2. In another embodiment, this invention relates to a pharmaceutical composition comprising Compound A wherein not more than 80% by weight, or not more than 70% by weight, or not more than 60% by weight, or not more than 50% by weight, or not more than 40% by weight, or not more than 30% by weight, or not more than 20% by weight, or not more than 10% by weight of Compound A is present in a form other than Compound A—Non-solvated Form 2. In another embodiment, this invention relates to a pharmaceutical composition comprising Compound A wherein not more than 5% by weight, or not more than 4% by weight, or not more than 3% by weight, or not more than 2% by weight, or not more than 1% by weight, or not more than 0.5% by weight, or not more than 0.2% by weight, or not more than 0.1% by weight of Compound A is present in a form other than Compound A—Non-solvated Form 2.

In another embodiment, this invention relates to a pharmaceutical composition comprising Compound A wherein not more than 90% by weight of Compound A is present in a form other than Compound A—Non-solvated Form 3. In another embodiment, this invention relates to a pharmaceutical composition comprising Compound A wherein not more than 80% by weight, or not more than 70% by weight, or not more than 60% by weight, or not more than 50% by weight, or not more than 40% by weight, or not more than 30% by weight, or not more than 20% by weight, or not more than 10% by weight of Compound A is present in a form other than Compound A—Non-solvated Form 3. In another embodiment, this invention relates to a pharmaceutical composition comprising Compound A wherein not more than 5% by weight, or not more than 4% by weight, or not more than 3% by weight, or not more than 2% by weight, or not more than 1% by weight, or not more than 0.5% by weight, or not more than 0.2% by weight, or not more than 0.1% by weight of Compound A is present in a form other than Compound A—Non-solvated Form 3.

### Experimentals

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention. Unless otherwise noted, reagents are commercially available or are prepared according to procedures in the literature. The symbols and conventions used in the descriptions of processes, schemes, and examples are consistent with those used in the contemporary scientific literature, for example, the Journal of the American Chemical Society or the Journal of Biological Chemistry.

In the Examples:

Chemical shifts are expressed in parts per million (ppm) units. Coupling constants (J) are in units of hertz (Hz). Splitting patterns describe apparent multiplicities and are designated as s (singlet), d (doublet), t (triplet), q (quartet), dd (double doublet), dt (double triplet), dq (double quartet), m (multiplet), br (broad).

Flash column chromatography was performed on silica gel.

The naming program used was ChemBioDraw° Ultra 12.0.

### Abbreviations

18-crown-6 1,4,7,10,13,16-hexaoxacyclooctadecane
n-BuLi n-butyllithium
CDCl₃ chloroform-d
CD₃OD methanol-d₄
Cs₂CO₃ cesium carbonate
DCM dichloromethane
EA ethyl acetate
ES-LCMS electrospray liquid chromatography-mass spectrometry
EtOH ethanol
g gram(s)
h hour(s)
HCl hydrochloric acid
H₂SO₄ sulfuric acid
H₂O water
KOAc potassium acetate
KOH potassium hydroxide
LCMS liquid chromatography-mass spectrometry
LiOH—H₂O lithium hydroxide hydrate
MeCN acetonitrile
MeOH methanol
mg milligram(s)
MgSO₄ magnesium sulfate
min minute(s)
mL milliliter(s)
mmol millimole(s)
N₂ nitrogen gas
NaCN sodium cyanide
NaHCO₃ sodium bicarbonate
NaOH sodium hydroxide
Na₂SO₄ sodium sulphate
NBS N-bromosuccinimide
NH₄Cl ammonium chloride
NMR nuclear magnetic resonance
PdCl₂(dppf) 1,1′-bis(diphenylphosphino)ferrocene]dichloropalladium(II)
PE petroleum ether
PMB p-methoxybenzyl
rt room temperature
TBME tent-butyl methyl ether
TFA trifluoroacetic acid
THF tetrahydrofuran
TLC thin layer chromotrography
T₃P® propylphosphonic anhydride

### EXAMPLE 1

### Preparation of: 2-(4-(4-Ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A)

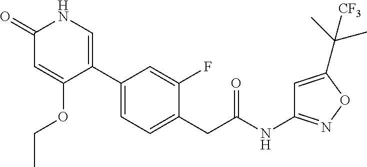 [SMILES: CCOc1cc(=O)[nH]cc1-c1ccc(CC(=O)Nc2cc(C(C)(C)C(F)(F)F)on2)c(F)c1]

### Step 1: 5,5,5-Trifluoro-4,4-dimethyl-3-oxopentanenitrile

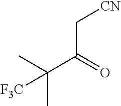

To a mixture of MeCN (13.9 mL, 264 mmol) in THF (500 mL) cooled to −78° C. was added n-BuLi (106 mL, 264 mmol). The mixture was stirred at −30° C. for 0.5 h. Then to the mixture was added methyl 3,3,3-trifluoro-2,2-dimethylpropanoate (30 g, 176 mmol) dropwise. The mixture was stirred at 25° C. for 10 h. The mixture was quenched with aqueous NH₄Cl (50 mL), extracted with EA (300 mL×3). The organic layer was dried over Na₂SO₄, filtered and concentrated to yield a crude product of a yellow oil of 5,5,5-trifluoro-4,4-dimethyl-3-oxopentanenitrile (22 g, 122.9 mmol, 70%): ¹H NMR (400 MHz, CDCl₃) δ 3.75 (s, 2H), 1.41 (s, 6H).

### Step 2: 5-(1,1,1-Trifluoro-2-methylpropan-2-yl)isoxazol-3-amine

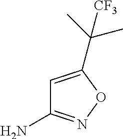

To a mixture of hydroxylamine hydrochloride (23.2 g, 336 mmol) in water (300 mL) cooled to 0° C. was added NaHCO₃ (30 g, 351 mmol) and pH=7.5 adjusted. Then to the mixture was added a solution of 5,5,5-trifluoro-4,4-dimethyl-3-oxopentanenitrile (30 g, 167.4 mmol) in MeOH (40 mL). The mixture was stirred at 65° C. for 15 h. After cooled, the mixture was acidified with concentrated HCl to pH=1 and then refluxed for 2 h. After cooling to rt, the mixture was neutralized by 4 M NaOH to pH=8. The mixture was extracted with EA (300 mL×2). The organic layer was dried over Na₂SO₄, filtered and concentrated. The crude material was purified by silica column chromatography (PE/EA=8:1-3:1). All fractions found to contain product by TLC (PE/EA=2:1, R_{f}=0.6) were combined and concentrated to yield a red solid of 5-(1,1,1-trifluoro-2-methylpropan-2-yl)isoxazol-3-amine (19.5 g, 100.5 mmol, 60%): ¹H NMR (400 MHz, CDCl₃) δ 5.79 (s, 1H), 3.96 (s., 2H), 1.53 (s, 6H); ES-LCMS m/z: 195 (M+H).

### Step 3: 2-Chloro-4-ethoxypyridine

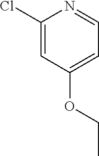

To a mixture of 2-chloro-4-nitropyridine (170 g, 1070 mmol) in THF (2 L) was added sodium ethanolate (109.45 g, 1610 mmol) slowly at 0° C. The mixture was stirred at 25° C. for 12 h. LCMS and TLC analysis (PE/EA=5:1, R_{f}=0.6) showed the reaction was finished. The mixture was filtered, and most of the filtrate solvent was removed by reduced pressure. The mixture was quenched with water and extracted with EA, the organic layer was washed with brine, and then concentrated. Another six batches were prepared following the same procedure to give 2-chloro-4-ethoxypyridine (1100 g, 7.01 mol, 92.4%): ¹H NMR (400 MHz, CD₃OD) δ 8.15 (d, J=6.0 Hz, 1H), 6.99 (d, J=2.0 Hz, 1H), 6.91-6.89 (m, 1H), 4.16-4.14 (m, 2H), 1.41-1.38 (m, 3H); ES-LCMS m/z: 158.1 (M+H).

### Step 4: 5-Bromo-2-chloro-4-ethoxypyridine

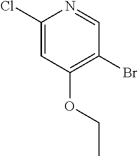

2-Chloro-4-ethoxypyridine (100 g, 634.5 mmol) was added to H₂50₄ (500 mL) slowly. NBS (124.2 g, 698.0 mmol) was then added to the above reaction mixture at rt. The mixture was stirred at 80° C. for 3 h. TLC analysis (PE/EA=10:1, R_{f}=0.5) showed the reaction was finished. The reaction mixture was poured into ice-water (2000 mL), extracted with EA, and then concentrated. Another ten batches were prepared following the same procedure. The combined crude product was purified by flash column chromatography to give 5-bromo-2-chloro-4-ethoxypyridine (670 g, 2.84 mol, 40.0%): ¹H NMR (400 MHz, CD₃OD): δ 8.31 (s, 1H), 7.14 (s, 1H), 4.32-4.10 (m, 2H), 1.58-1.35 (m, 3H); ES-LCMS m/z: 236.0, 238.0 (M, M+2H).

### Step 5: 5-Bromo-4-ethoxy-2-((4-methoxybenzyl)oxy)pyridine

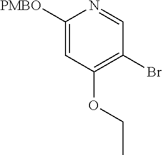

To a mixture of 5-bromo-2-chloro-4-ethoxypyridine (75 g, 317.1 mmol) in toluene (500 mL) was added (4-methoxyphenyl)methanol (52.6 g, 380.6 mmol), KOH (35.6g, 634.3 mmol) and 18-crown-6 (8.4 g, 31.2 mmol) at rt. The reaction mixture was stirred at 120° C. for 2 h. The mixture was extracted with TBME, washed with brine, and concentrated. Another eight batches were prepared following the same procedure. The combined crude product was purified by flash column chromatography (PE/EA=10:1, R_{f}=0.5) to give 5-bromo-4-ethoxy-2-((4-methoxybenzyl)oxy)pyridine (650 g, 1.99 mol, 70.0%): ¹H NMR (400 MHz, CD₃OD) δ 8.05 (s, 1H), 7.33 (d, J=8.6 Hz, 2H), 6.90-6.84 (m, 2H), 6.38 (s, 1H), 5.20 (s, 2H), 4.16-4.05 (m, 2H), 3.77 (s, 3H), 1.43 (q, J=6.8 Hz, 3H); ES-LCMS m/z: 338.3 (M+2H).

### Step 6: 2-(4-Bromo-2-fluorophenyl)acetonitrile

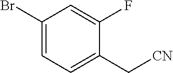 [SMILES: N#CCc1ccc(Br)cc1F]

To a solution of 4-bromo-1-(bromomethyl)-2-fluorobenzene (500 g, 1.87 mol) in EtOH (2.2 L) stirred under N₂ at 20° C. was added NaCN (93 g, 1.90 mmol) in one charge. The reaction mixture was stirred at 60° C. for 12 h. Then the solution was concentrated and distributed between DCM (2000 mL) and saturated NaHCO₃ solution (1800 mL). Another batch was prepared following the same procedure. Then the two batches were combined. The combined organic extract was washed with brine, dried over MgSO₄, filtered and concentrated to provide 2-(4-bromo-2-fluorophenyl)acetonitrile (794 g, 99%): ¹H NMR (400 MHz, CDCl₃) δ 7.38-7.27 (m, 3H), 3.72 (s, 2H).

### Step 7: 2-(4-Bromo-2-fluorophenyl)acetic acid

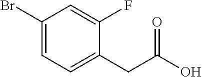 [SMILES: O=C(O)Cc1ccc(Br)cc1F]

To a solution of 2-(4-bromo-2-fluorophenyl)acetonitrile (397 g, 1.82 mol) in MeOH (500 mL) stirred under N₂ at 20° C. was added NaOH (2.22 L, 2.5M, 5.56 mol) solution in one charge. The reaction mixture was stirred at 80° C. for 5 h. Then the solution was concentrated and neutralized with conc. HCl to pH=5 with stirring. Then the solution was extracted with EA (1.5 L×2). Another two batches were prepared following the same procedure. Then the three batches were combined. The combined organic extract was washed with brine, dried over Na₂SO₄, filtered and concentrated in vacuo to give the pure 2-(4-bromo-2-fluorophenyl)acetic acid (1200 g, 92%): TLC (PE/EA=5:1, R_{f}=0.2); ¹H NMR (400 MHz, CDCl₃) δ 7.24 (br. s., 1H), 7.12 (t, J=7.9 Hz, 1H), 3.65 (s, 2H).

### Step 8: Methyl 2-(4-bromo-2-fluorophenyl)acetate

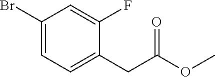

To a solution of 2-(4-bromo-2-fluorophenyl)acetic acid (260 g, 1.13 mol) in MeOH (2 L) was added H₂SO₄ (30 mL) at rt. The solution was heated to reflux overnight. Then the solvent was concentrated and the residue was distributed between EA and saturated NaHCO₃ solution. The organic extract was washed with brine, dried over Na₂SO₄, filtered and concentrated. Another batch was prepared following the same procedure. Then the two batches were combined to provide methyl 2-(4-bromo-2-fluorophenyl)acetate (520 g, 94%). TLC (PE/EA=10:1, R_{f}=0.7). ¹H NMR (400 MHz, CDCl₃) δ 7.25-7.20 (m, 2H), 7.14 (t, J=8.0 Hz, 1H), 3.70 (s, 3H), 3.62 (s, 2H).

### Step 9: Methyl 2-(2-fluoro-4-(4,4,5,5-tetramethyl-1,3,2-dioxaborolan-2-yl)phenyl)acetate

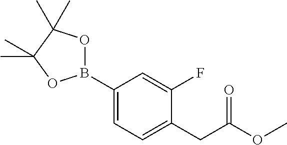

To a solution of methyl 2-(4-bromo-2-fluorophenyl)acetate (260 g,1.05 mol) and 4,4,4′,4′,5,5,5′,5′-octamethyl-2,2′-bi(1,3,2-dioxaborolane) (320 g, 1.26 mol) in 1,4-dioxane (2 L) was added KOAc (206 g, 2.10 mol) and PdCl₂(dppf) (23 g, 0.03 mol) at rt. The solution was heated to reflux for 4 h under N₂. Then the solution was filtered and the filtrate was concentrated in vacuo to give the crude product. Another batch was prepared following the same procedure. Then the two batches were combined and purified by flash column chromatography (PE/EA=30:1 to 10:1). All fractions found to contain product by TLC (PE/EA=10:1, R_{f}=0.5) were combined and concentrated to yield methyl 2-(2-fluoro-4-(4,4,5,5-tetramethyl-1,3,2-dioxaborolan-2-yl)phenyl)acetate (560 g, 90%) as a light yellow oil: ¹H NMR (400 MHz, CDCl₃) δ 7.54 (d, J=7.5 Hz, 1H), 7.49 (d, J=10.0 Hz, 1H), 7.31-7.26 (m, 1H), 3.73 (s, 2H), 1.34 (s, 12H), 1.27 (s, 3H); ES-LCMS m/z 295.2 (M+H).

### Step 10: Methyl 2-(4-(4-ethoxy-6-((4-methoxybenzyl)oxy)pyridin-3-yl)-2-fluorophenyl)acetate

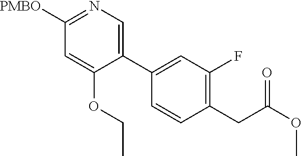

To a solution of 5-bromo-4-ethoxy-2-((4-methoxybenzyl)oxy)pyridine (175 g, 519 mmol) in 1,4-dioxane (1200 mL) and H₂O (300 mL) was added methyl 2-(2-fluoro-4-(4,4,5,5-tetramethyl-1,3,2-dioxaborolan-2-yl)phenyl)acetate (167 g, 569 mmol), PdCl₂(dppf) (25 g, 5.19 mmol) and Cs₂CO₃ (337 g, 1038 mmol) under a N₂ atmosphere. The mixture was refluxed for 2 h. TLC analysis (PE/EA=5:1, R_{f}=0.3) showed the reaction was finished. The mixture was extracted with EA/H₂O (2 L) to give the oil layer, which was dried over Na₂SO₄, filtered, concentrated. Another two batches were prepared following the same procedure. The combined crude product was purified by flash column chromatography (PE/EA=5:1, R_{f}=0.3) to give 5-bromo-4-ethoxy-2-((4-methoxybenzyl)oxy)pyridine (630 g, 1.48 mol, 90.0%): ¹H NMR (400 MHz, CD₃OD) δ 7.94 (s, 1H), 7.36 (d, J=8.8 Hz, 2H), 7.32-7.22 (m, 3H), 6.90 (d, J=8.8 Hz, 2H), 6.43 (s, 1H), 5.26 (s, 2H), 4.11 (d, J=6.8 Hz, 2H), 3.78 (s, 3H), 3.72 (s, 2H), 3.70 (s, 3H), 1.36 (t, J=7.0 Hz, 3H); ES-LCMS m/z: 426.1 (M+H).

### Step 11: 2-(4-(4-Ethoxy-6-((4-methoxybenzyl)oxy)pyridin-3-yl)-2-fluorophenyl)acetic acid

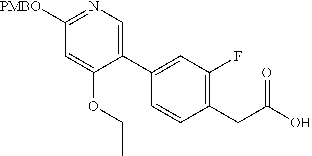

To a solution of methyl 2-(4-(4-ethoxy-6-((4-methoxybenzyl)oxy)pyridin-3-yl)-2-fluorophenyl)acetate (210 g, 519 mmol) in THF (500 mL) was added LiOH—H₂O (52 g, 1230 mmol) in H₂O (700 mL). The mixture was stirred at 60° C. overnight. TLC analysis

(PE/EA=5:1, Rf=0.3) showed the reaction was finished. The mixture was concentrated, and adjusted with HCl (1 N) to pH=7. Another two batches were prepared following the same procedure. Then the combined crude product was filtered, the solid was washed with water and dried to give 2-(4-(4-ethoxy-6-((4-methoxybenzyl)oxy)pyridin-3-yl)-2-fluorophenyl)acetic acid (550 g, 1.34 mol, 93.0%): ¹H NMR (400 MHz, CD₃OD): δ 7.94 (s, 1H), 7.41-7.28 (m, 3H), 7.24 (d, J=9.5 Hz, 2H), 6.91 (d, J=8.6 Hz, 2H), 6.44 (s, 1H), 5.26 (s, 2H), 4.11 (q, J=6.9 Hz, 2H), 3.78 (s, 3H), 3.67 (s, 2H), 1.36 (t, J=7.0 Hz, 3H); ES-LCMS m/z: 412.1 (M+H).

### Step 12: 2-(4-(4-Ethoxy-6-((4-methoxybenzyl)oxy)pyridin-3-yl)-2-fluorophenyl)-N-(5-(1,1,1-trifluoro-2-methylpropan-2-yl)isoxazol-3-yl)acetamide

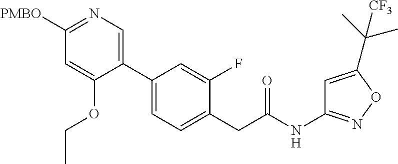

To a mixture of 2-(4-(4-ethoxy-6-((4-methoxybenzyl)oxy)pyridin-3-yl)-2-fluorophenyl)acetic acid (55.1 g, 134 mmol) and 5-(1,1,1-trifluoro-2-methylpropan-2-yl)isoxazol-3-amine (26 g, 134 mmol) in pyridine (500 mL) was added T₃P® (137.5 mL, 134 mmol) dropwise and stirred at 25° C. for 1 h. After TLC analysis showed the starting material was consumed completely, the mixture was poured into stirring cold water (1 L). The mixture was stirred for 0.5 h and then let stand for 10 h. The solid was filtered, washed with H₂O (200 mL×3) and TBME (200 mL×2) and dried in vacuo to give an off-white solid of 2-(4-(4-ethoxy-6-((4-methoxybenzyl)oxy)pyridin-3-yl)-2-fluorophenyl)-N-(5-(1,1, 1-trifluoro-2-methylpropan-2-yl)i soxazol-3 -yl)acetami de (65 g, 100 mmol, 74%): ¹H NMR (400 MHz, CD₃OD) δ 7.94 (s, 1H), 7.40-7.32 (m, 3H), 7.26 (d, J=9.6 Hz, 2H), 6.90 (d, J=8.8 Hz, 3H), 6.43 (s, 1H), 5.26 (s, 2H), 4.11 (q, J=7.2 Hz, 2H), 3.81 (s, 2H), 3.78 (s, 3H), 1.56 (s, 6H), 1.35 (t, J=7.2 Hz, 3H); ES-LCMS m/z: 588 (M+H).

### Step 13: 2-(4-(4-Ethoxy-6-oxo-1,6-dihydropyridin-3-yl)-2-fluorophenyl)-N-(5-(1,1,1-trifluoro-2-methylpropan-2-yl)isoxazol-3-yl)acetamide

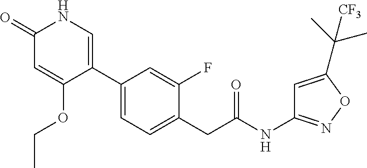 [SMILES: CCOc1cc(=O)[nH]cc1-c1ccc(CC(=O)Nc2cc(C(C)(C)C(F)(F)F)on2)c(F)c1]

To a suspension of 2-(4-(4-ethoxy-6-((4-methoxybenzyl)oxy)pyridin-3-yl)-2-fluorophenyl)-N-(5-(1,1,1-trifluoro-2-methylpropan-2-yl)isoxazol-3-yl)acetamide (100 g, 170 mmol) in DCM (1 L) was added TFA (80 mL, 1077 mmol) dropwise. The mixture was stirred at 25° C. for 2 h. The mixture was then concentrated. To the residue was added H₂O (500 mL) dropwise and then neutralized with saturated Na₂CO₃ solution to adjust pH=7.5. The precipitate was filtered, washed with H₂O (350 mL×3) and dried in vacuo. To the solid was added PE/EA (3:1, v/v, 300 mL) and stirred for 0.5 h. The solid was filtered and washed with PE/EA (3:1, v/v, 100 mL×2). The solid was redissolved in DCM/MeOH (20:1, v/v, 1.5 L) and then concentrated in vacuo to a minimal amount of solvent (about 150 mL). The solid was filtered, washed with MeCN (50 mL×2) and dried in vacuo. The residual solid was added to EtOH (2.5 L) and heated to 80° C. After the solid was dissolved completely, the mixture was concentrated in vacuo to give a white solid of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (61.4 g, 131 mmol, 77%): ¹H NMR (400 MHz, CD₃OD) δ 7.40-7.30 (m, 2H), 7.25-7.18 (m, 2H), 6.88 (s, 1H), 5.98 (s, 1H), 4.11 (q, J=7.2 Hz, 2H), 3.81 (s, 2H), 1.56 (s, 6H), 1.37 (t, J=7.2 Hz, 3H); ES-LCMS m/z: 468 (M+H).

### EXAMPLE 2

### Preparation of: A crystalline monohydrate of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Monohydrate)

2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (407 mg) was added to a 20 mL vial followed by water (8.1 mL). The suspension was heated to 40° C. and cycled from 40° C. to 5° C. in 1 h blocks overnight with stirring. The solids were filtered and air-dried for 20 min. The yield of the crystalline product was 373 mg (91.6%).

The X-ray powder diffraction (XRPD) pattern of this material (Compound A—Monohydrate) is shown in FIG. 1 and a summary of the diffraction angles and d-spacings is given in Table I below. The XRPD analysis was conducted on a PANanalytical X'Pert Pro Diffractometer on Si zero-background wafers. The acquisition conditions included: Cu K_{α} radiation, generator tension: 45 kV, generator current: 40 mA, step size: 0.02° 2θ.

TABLE I

| Diff. Angle [°2θ] | d-spacing [Å] |
|---|---|
| 10.10932 | 8.7501 |
| 10.74198 | 8.23614 |
| 11.54514 | 7.66491 |
| 13.22787 | 6.6934 |
| 13.945 | 6.35076 |
| 14.29196 | 6.19735 |
| 16.6849 | 5.31353 |
| 17.07666 | 5.19251 |
| 17.6015 | 5.03884 |
| 18.2858 | 4.85179 |
| 18.41953 | 4.81687 |
| 18.9332 | 4.68733 |
| 20.28906 | 4.37704 |
| 20.6827 | 4.29462 |
| 21.3928 | 4.15365 |
| 21.56444 | 4.12097 |
| 22.04311 | 4.03256 |
| 23.22829 | 3.82942 |
| 23.89207 | 3.72451 |
| 24.87764 | 3.57914 |
| 25.1863 | 3.53598 |
| 26.349 | 3.38253 |
| 26.59132 | 3.35225 |
| 27.37473 | 3.25807 |
| 28.61497 | 3.11962 |
| 29.27541 | 3.05073 |
| 30.04912 | 2.97391 |
| 30.68794 | 2.91345 |
| 31.24132 | 2.86309 |
| 32.56886 | 2.74936 |
| 34.32998 | 2.61225 |
| 35.89718 | 2.50171 |
| 38.51498 | 2.33749 |
| 39.3974 | 2.28715 |

The Raman spectrum of the title compound was recorded on a Nicolet NXR 9650 FT-Raman Spectrometer, at 4 cm⁻¹ resolution with excitation from a Nd:YVO4 laser (λ=1064 nm). The Raman spectrum of this material is shown in FIG. 2 with major peaks observed at 422, 450, 489, 516, 545, 575, 669, 700, 716, 733, 774, 818, 894, 918, 963, 989, 1032, 1112, 1174, 1241, 1296, 1334, 1428, 1463, 1484, 1506, 1532, 1566, 1629, 1645, 1721, 2930, 2990, and 3087 cm⁻¹.

The differential scanning calorimetry (DSC) thermogram of the title compound was recorded on a TA Instruments Q100 Differential Scanning calorimeter equipped with an autosampler and a refrigerated cooling system under 40 mL/min N₂ purge and is shown in FIG. 3. The experiments were conducted using a heating rate of 15° C./min in a crimped aluminum pan. The DSC thermogram of Compound A—Monohydrate exhibits a double endotherm with an onset temperature of about 139° C. followed by a single endotherm with an onset temperature of about 241° C. A person skilled in the art would recognize that the onset temperature of the endotherm may vary depending on the experimental conditions.

The thermogravimetric analysis (TGA) thermogram of the title compound was recorded on a TA Instruments Q500 Thermogravimetric Analyzer and is shown in FIG. 4. The experiments were conducted with 40 mL/min N₂ flow and a heating rate of 15° C./min. The TGA thermogram of Compound A—Monohydrate exhibits a loss of about 3.7% water (1.0 eq) from 75-160° C.

Drying of Compound A—Monohydrate in a vacuum oven at 50° C. with a nitrogen bleed for about 17 hours resulted in no change to the water content by TGA and no change in form by Raman or XRPD was observed.

### EXAMPLE 3

### Preparation of: A crystalline non-solvated form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 1)

2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (160 mg) was added to a 4 mL vial followed by MeCN (3.2 mL). The suspension was heated to 40° C. and cycled from 40° C. to 5° C. in 1 h blocks overnight with stirring. The solids were filtered and air-dried for 20 min. The sample was dried at 50° C. in a vaccum oven with nitrogen bleed for 4 h. The yield of the crystalline product was 152 mg (95.0%).

The X-ray powder diffraction (XRPD) pattern of this material (Compound A—Non-solvated Form 1) is shown in FIG. 5 and a summary of the diffraction angles and d-spacings is given in Table II below. The XRPD analysis was conducted on a PANanalytical X'Pert Pro Diffractometer on Si zero-background wafers. The acquisition conditions included: Cu K_{α} radiation, generator tension: 45 kV, generator current: 40 mA, step size: 0.02° 2θ.

TABLE II

| Diff. Angle [°2θ] | d-spacing [Å] |
|---|---|
| 4.505409 | 19.61323 |
| 5.008904 | 17.64279 |
| 5.987154 | 14.76212 |
| 7.945965 | 11.12687 |
| 9.253216 | 9.55765 |
| 10.03458 | 8.8151 |
| 11.20182 | 7.89905 |
| 13.06056 | 6.77876 |
| 13.32806 | 6.64331 |
| 13.77428 | 6.42908 |
| 14.97775 | 5.9151 |
| 15.52406 | 5.70815 |
| 16.63975 | 5.32785 |
| 17.06157 | 5.19707 |
| 18.23162 | 4.86609 |
| 18.65978 | 4.75539 |
| 18.99956 | 4.67111 |
| 19.66394 | 4.51476 |
| 20.22573 | 4.39061 |
| 20.73202 | 4.28452 |
| 21.56666 | 4.12055 |
| 22.61362 | 3.93209 |
| 23.27202 | 3.82232 |
| 23.82079 | 3.73549 |
| 24.26295 | 3.66841 |
| 25.95131 | 3.43345 |
| 26.57554 | 3.3542 |
| 27.23522 | 3.27444 |
| 28.05712 | 3.18036 |
| 28.68344 | 3.11233 |
| 29.14829 | 3.06374 |
| 30.27289 | 2.95244 |
| 31.26402 | 2.86107 |
| 35.60345 | 2.52168 |

The Raman spectrum of the title compound was recorded on a Nicolet NXR 9650 FT-Raman Spectrometer, at 4 cm⁻¹ resolution with excitation from a Nd:YVO4 laser (λ=1064 nm). The Raman spectrum of this material is shown in FIG. 6 with major peaks observed at 450, 544, 566, 668, 726, 771, 819, 898, 978, 1035, 1110, 1176, 1242, 1273, 1329, 1424, 1470, 1484, 1511, 1534, 1626, 1681, 2930, 2999, and 3093 cm⁻¹.

The differential scanning calorimetry (DSC) thermogram of the title compound was recorded on a TA Instruments Q100 Differential Scanning calorimeter equipped with an autosampler and a refrigerated cooling system under 40 mL/min N₂ purge and is shown in FIG. 7. The experiments were conducted using a heating rate of 15° C./min in a crimped aluminum pan. The DSC thermogram of Compound A—Non-solvated Form 1 exhibits small thermal events around about 130-160° C. followed by endotherms with an onset temperature of about 236° C. and about 241° C. A person skilled in the art would recognize that the onset temperature of the endotherm may vary depending on the experimental conditions.

The thermogravimetric analysis (TGA) thermogram of the title compound was recorded on a TA Instruments Q500 Thermogravimetric Analyzer and is shown in FIG. 8. The experiments were conducted with 40 mL/min N₂ flow and a heating rate of 15° C./min. The TGA thermogram of Compound A—Non-solvated Form 1 exhibits a weight loss of about 0.6% from 75-160° C.

### EXAMPLE 4

### Preparation of: A crystalline non-solvated form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 2)

2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Monohydrate) was dehydrated by heating to 160° C. and holding for 5 min.

The X-ray powder diffraction (XRPD) pattern of this material (Compound A—Non-solvated Form 2) is shown in FIG. 9 and a summary of the diffraction angles and d-spacings is given in Table III below. The XRPD analysis was conducted on a PANanalytical X'Pert Pro Diffractometer on Si zero-background wafers. The acquisition conditions included: Cu K_{α} radiation, generator tension: 45 kV, generator current: 40 mA, step size: 0.02° 2θ.

TABLE III

| Diff. Angle [°2θ] | d-spacing [Å] |
|---|---|
| 6.379807 | 13.85442 |
| 12.68489 | 6.97866 |
| 14.20764 | 6.23395 |
| 15.41767 | 5.7473 |
| 16.06748 | 5.5163 |
| 17.17094 | 5.16421 |
| 17.94964 | 4.94189 |
| 18.93004 | 4.6881 |
| 19.62118 | 4.5245 |
| 20.14145 | 4.40879 |
| 21.22904 | 4.18532 |
| 21.92363 | 4.05426 |
| 22.76014 | 3.90711 |
| 23.6936 | 3.75525 |
| 24.65676 | 3.6107 |
| 25.5554 | 3.48574 |
| 26.59859 | 3.35135 |
| 28.70476 | 3.11006 |
| 29.52049 | 3.02595 |
| 32.29181 | 2.77231 |
| 34.89922 | 2.57093 |

The Raman spectrum of the title compound was recorded on a Nicolet NXR 9650 FT-Raman Spectrometer, at 4 cm⁻¹ resolution with excitation from a Nd:YVO4 laser (λ=1064 nm). The Raman spectrum of this material is shown in FIG. 10 with major peaks observed at 417, 451, 486, 544, 576, 669, 697, 716, 730, 771, 821, 900, 964, 986, 1035, 1109, 1175, 1243, 1265, 1300, 1336, 1430, 1465, 1487, 1527, 1631, 1640, 1726, 2919, 2949, 2997, and 3082 cm⁻¹.

The differential scanning calorimetry (DSC) thermogram of the title compound was recorded on a TA Instruments Q100 Differential Scanning calorimeter equipped with an autosampler and a refrigerated cooling system under 40 mL/min N₂ purge and is shown in FIG. 11. The experiments were conducted using a heating rate of 15° C./min in a crimped aluminum pan. The DSC thermogram of Compound A—Non-solvated Form 2 exhibits a single endotherm with an onset temperature of about 240° C. A person skilled in the art would recognize that the onset temperature of the endotherm may vary depending on the experimental conditions.

### EXAMPLE 5

### Preparation of: A crystalline non-solvated form of 2-(4-(4-ethoxy-6-oxo-1,6-dihydropyridin-3-yl)-2-fluorophenyl)-N-(5-(1,1,1-trifluoro-2-methylpropan-2-yl)isoxazol-3-yl)acetamide (Compound A—Non-solvated Form 3)

2-(4-(4-ethoxy-6-oxo-1,6-dihydropyridin-3 -yl)-2-fluorophenyl)-N-(5-(1,1,1-trifluoro-2-methylpropan-2-yl)isoxazol-3-yl)acetamide (508.9 mg) was added to a 20 mL vial followed by MeOH (10.0 mL). The suspension was heated to 40° C. and cycled from 40° C. to 5° C. in 1 h blocks overnight with stirring. The solids were filtered and air-dried for 20 min. The yield of the crystalline product was 337.8 mg (66.4%).

The X-ray powder diffraction (XRPD) pattern of this material (Compound A—Non-solvated Form 3) is shown in FIG. 12 and a summary of the diffraction angles and d-spacings is given in Table IV below. The XRPD analysis was conducted on a PANanalytical X'Pert Pro Diffractometer on Si zero-background wafers. The acquisition conditions included: Cu K_{α} radiation, generator tension: 45 kV, generator current: 40 mA, step size: 0.02° 2θ.

TABLE IV

| Diff. Angle [°2θ] | d-spacing [Å] |
|---|---|
| 9.613192 | 9.20054 |
| 10.95047 | 8.07978 |
| 11.72278 | 7.54916 |
| 13.77348 | 6.42945 |
| 14.26174 | 6.21042 |
| 15.31682 | 5.78491 |
| 16.62671 | 5.332 |
| 17.2211 | 5.14928 |
| 17.51262 | 5.06422 |
| 18.75647 | 4.73109 |
| 19.26456 | 4.60744 |
| 20.32107 | 4.37022 |
| 21.05053 | 4.2204 |
| 21.42294 | 4.14787 |
| 21.99328 | 4.04158 |
| 23.00655 | 3.86582 |
| 23.60721 | 3.7688 |
| 24.54124 | 3.62744 |
| 25.84386 | 3.44748 |
| 26.16735 | 3.40559 |
| 27.44447 | 3.24995 |
| 27.74445 | 3.21549 |
| 28.5692 | 3.12451 |
| 29.55222 | 3.02278 |
| 30.81036 | 2.89975 |
| 30.9598 | 2.88848 |
| 31.36629 | 2.85197 |
| 32.31128 | 2.77069 |
| 33.25038 | 2.69455 |
| 35.93842 | 2.49894 |
| 39.20647 | 2.29784 |

The Raman spectrum of the title compound was recorded on a Nicolet NXR 9650 FT-Raman Spectrometer, at 4 cm⁻¹ resolution with excitation from a Nd:YVO4 laser (λ=1064 nm). The Raman spectrum of this material is shown in FIG. 13 with major peaks observed at 454, 493, 572, 639, 728, 769, 819, 841, 923, 978, 1037, 1109, 1190, 1239, 1287, 1331, 1429, 1464, 1485, 1509, 1542, 1631, 1714, 2951, 2994, 3078, and 3093 cm⁻¹.

The differential scanning calorimetry (DSC) thermogram of the title compound was recorded on a TA Instruments Q100 Differential Scanning calorimeter equipped with an autosampler and a refrigerated cooling system under 40 mL/min N₂ purge and is shown in FIG. 14. The experiments were conducted using a heating rate of 15° C./min in a crimped aluminum pan. The DSC thermogram of Compound A—Non-solvated Form 3 exhibits a single endotherm with an onset temperature of about 248° C. A person skilled in the art would recognize that the onset temperature of the endotherm may vary depending on the experimental conditions.

The thermogravimetric analysis (TGA) thermogram of the title compound was recorded on a TA Instruments Q500 Thermogravimetric Analyzer and is shown in FIG. 15. The experiments were conducted with 40 mL/min N₂ flow and a heating rate of 15° C./min.

### Biological Assays

The compound of the present invention was tested for RET kinase inhibitory activity in a RET kinase enzyme assay, a cell-based mechanistic assay and a cell-based proliferation assay.

### RET Kinase Enzymatic Assay

Human RET kinase cytoplasmic domain (amino acids 658-1114 of accession number NP_000314.1) was expressed as an N-terminal GST-fusion protein using a baculovirus expression system. GST-RET was purified using glutathione sepharose chromatography. The RET kinase enzymatic assay was performed in a total volume of 10 uL with increasing concentrations of RET kinase inhibitor as a singlet in a 384 well format as follows: RET inhibitor compound plates are prepared by adding 100 nL of RET inhibitor at different concentrations to a 384-well plate. 5 μL/well of a 2× enzyme mix (50 mM HEPES (4-(2-hydroxyethyl)-1-piperazineethanesulfonic acid); 1 mM CHAPS (3-[(3-cholamidopropyl)dimethylammonio]-1-propanesulfonate); 0.1 mg/mL BSA (bovine serum albumin); 1 mM DTT (dithiothreitol); 0.2 nM RET kinase) was added to the 384-well plate and incubated for 30 minutes at 23° C. 5 μL/well of a 2× substrate mix (50 mM HEPES; 1 mM CHAPS; 0.1 mg/mL BSA; 20 μM adenosine triphosphate; 20 mM MgCl₂ and 1 μM biotinylated peptide substrate) was added and incubated for 1 hour at 23° C. 10 μL/well of 2× stop/detection mix (50 mM HEPES; 0.1% BSA; 800 mM Potassium Fluoride; 50 mM EDTA (Ethylenediaminetetraacetic acid); 200× dilution of Europium Cryptate labeled anti-phosphotyrosine antibody; 62.5 nM Streptavidin-XL665) incubated for 1 hour at 23° C. and read on a Homogenous Time-Resolved Fluorescence reader. IC₅₀s were fitted using GraphPad Prism to a sigmoidal dose response.

### RET Kinase Cell-Based Mechanistic Assay

The potency of the compound of the invention was tested for its ability to inhibit constitutive RET kinase phosphorylation in cell-based assay. TT cells (ATCC CRL-1803), a medullary thyroid cancer cell line with constitutively activated RET kinase, were maintained in 150 cm² dishes in F12 Kaighn's medium, 10% fetal bovine serum, 1× Glutamax, 1× non-essential amino acids, 1× Pen/Strep antibiotics at 37° C. in 5% carbon dioxide. 1.0E5 TT cells/well were plated in a 96-well cell culture plate and allowed to adhere overnight. TT cells were treated with different concentrations of RET inhibitor compounds for 2 h at 37° C. in 5% carbon dioxide, washed with ice cold PBS (phosphate buffered saline) and lysed by adding 200 μL of 25 mM Tris HCl pH 7.5; 2 mM EDTA; 150 mM NaCl; 1% sodium deoxycholate; 1% Triton X-100; 50 mM sodium beta glycerophosphate; 1 mM sodium orthovanadate; 1× phosphatase inhibitor cocktail #2 (Sigma #P5726); 1× phosphatase inhibitor cocktail #3 (Sigma #P0044) and 1× complete mini EDTA free protease inhibitor cocktail (Roche #4693159001), incubation at −80° C. for 10 minutes and thawed on ice. 100 μL of TT cell lysate was added to a 96-well plate overnight at 4° C. that had been coated overnight at 4° C. with 1:1,000 dilution of a rabbit anti-RET antibody (Cell Signaling #7032) blocked with 1× PBS; 0.05% Tween-20; 1% bovine serum albumin. Plates were washed 4× with 200 μL of 1× PBS; 0.05% Tween-20 and then 100 μL of a 1:1,000 dilution of an anti-phosphotyrosine detection antibody (Cell Signaling #7034) was added and incubated for 1 hour at 37° C. Plates were washed 4× with 200 μL of 1× PBS; 0.05% Tween-20 and then 100 μL of a 1:1,000 dilution of an anti-mouse immunoglobulin horse radish peroxidase conjugate antibody (Cell Signaling #7034) was added and incubated for 30 minutes at 37° C. Plates were washed 4× with 200 μL of 1× PBS; 0.05% Tween-20, 100 μL of TMB (3,3′, 5,5″-tetramethylbenzidine) substrate (Cell Signaling #7004) was added, incubated for 10 minutes at 37° C., 100 μL of Stop solution (Cell Signaling #7002) was added and absorbance read on a spectrophotometer at 450 nm. IC₅ₒs were fitted using GraphPad Prism to a sigmoidal dose response.

### RET Kinase Cell-Based Proliferation Assay

The potency of the compound of the invention was tested for its ability to inhibit cell proliferation and cell viability. TT cells (ATCC CRL-1803), a medullary thyroid cancer cell line with constitutively activated RET kinase, were maintained in 150 cm² dishes in F12 Kaighn's medium, 10% fetal bovine serum, 1× Glutamax, 1× non-essential amino acids, 1× Pen/Strep antibiotics at 37° C. in 5% carbon dioxide. 6.0E3 TT cells/well in 50 μL of media were added to a 96-well cell culture plate and allowed to adhere overnight. 50 μL of serially diluted RET inhibitor compounds were added to 96-well plate containing cultured TT cells and incubated at at 37° C. in 5% carbon dioxide for eight days. 50 μL of CellTiter-Glo (Promega #G-7573) was added, contents mixed for 1 minute on shaker followed by 10 minutes in the dark at 23° C. and the luminescence read by EnVision (PerkinElmer). IC₅₀s were fitted using GraphPad Prism to a sigmoidal dose response.

### Biological Data

The compound of Example 1 was tested in the RET assays described above and was found to be an inhibitor of RET. Data for the compound of Example 1 are listed below in Table V as follows: +=10 μM≧IC₅₀>100 nM; ++=100 nM≧IC₅₀>10 nM; +++=IC₅₀≦10 nM.

TABLE V

| | Human RET | Human RET | Human RET |
|---|---|---|---|
| | kinase | kinase cell-based | kinase cell-based |
| Example # | enzymatic IC₅₀ | mechanistic IC₅₀ | proliferation IC₅₀ |
| 1 | +++ | +++ | ++ |

### In Vivo Colonic Hypersensitivity Model

The efficacy of RET kinase inhibitor compounds can be evaluated in an in vivo model of colonic hypersensitivity (Hoffman, J. M., et al., Gastroenterology, 2012, 142:844-854).

1. A crystalline form of 2-(4-(4-ethoxy-6-oxo-1,6-dihydropyridin-3-yl)-2-fluorophenyl)-N-(5-(1,1,1-trifluoro-2-methylpropan-2-yl)i soxazol-3 -yl)acetamide.

2. The crystalline form according to claim 1, wherein the crystalline form is characterized by an X-ray powder diffraction (XRPD) pattern comprising at least three diffraction angles, when measured using Cu K_{α} radiation, selected from a group consisting of about 10.1, 10.7, 11.5, 13.2, 13.9, 14.3, 16.7, 17.1, 17.6, 18.3, 18.4, 18.9, 20.3, 20.7, 21.4, 21.6, 22.0, 23.2, 23.9, 24.9, 25.2, 26.3, 26.6, 27.4, 28.6, 29.3, 30.0, 30.7, 31.2, 32.6, 34.3, 35.9, 38.5, and 39.4 degrees 2θ.

3. The crystalline form according to claim 1, wherein the crystalline form is characterized by an X-ray powder diffraction (XRPD) pattern comprising at least three diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, 25.2, 26.3, 26.6, 28.6, 30.0, and 32.6 degrees 2θ.

4. The crystalline form according to claim 1, wherein the crystalline form is characterized by an X-ray powder diffraction (XRPD) pattern comprising at least three diffraction angles, when measured using Cu K_{α} radiation, selected from a group consisting of about 10.1, 10.7, 11.5, 13.9, 17.1, 18.3, 18.4, 20.3, 20.7, 21.4, 21.6, 22.0, 23.2, 23.9, 24.9, and 26.6 degrees 2θ.

5. The crystalline form according to claim 1, wherein the crystalline form is characterized by an X-ray powder diffraction (XRPD) pattern comprising diffraction angles, when measured using Cu K_{α} radiation, of about 13.9, 17.1, 18.3, 18.4, 21.4, 21.6, and 23.9 degrees 2θ.

6. The crystalline form according to claim 2, wherein the crystalline form is characterized by an X-ray powder diffraction (XRPD) pattern substantially in accordance with FIG. 1.

7. The crystalline form according to claim 1, wherein the crystalline form is characterized by an X-ray powder diffraction (XRPD) pattern comprising at least three diffraction angles, when measured using Cu K_{α} radiation, selected from a group consisting of about 4.5, 5.0, 6.0, 7.9, 9.3, 10.0, 11.2, 13.1, 13.3, 13.8, 15.0, 15.5, 16.6, 17.1, 18.2, 18.7, 19.0, 19.7, 20.2, 20.7, 21.6, 22.6, 23.3, 23.8, 24.3, 26.0, 26.6, 27.2, 28.1, 28.7, 29.1, 30.3, 31.3, and 35.6 degrees 2θ.

8-9. (canceled)

10. The crystalline form according to claim 1, wherein the crystalline form is characterized by an X-ray powder diffraction (XRPD) pattern comprising diffraction angles, when measured using Cu K_{α} radiation, of about 13.1, 13.3, 17.1, 18.2, 21.6, 23.3, and 23.8 degrees 2θ.

11. The crystalline form according to claim 7, wherein the crystalline form is characterized by an X-ray powder diffraction (XRPD) pattern substantially in accordance with FIG. 5.

12. The crystalline form according to claim 1, wherein the crystalline form is characterized by an X-ray powder diffraction (XRPD) pattern comprising at least three diffraction angles, when measured using Cu K_{α} radiation, selected from a group consisting of about 6.4, 12.7, 14.2, 15.4, 16.1, 17.2, 17.9, 18.9, 19.6, 20.1, 21.2, 21.9, 22.8, 23.7, 24.7, 25.6, 26.6, 28.7, 29.5, 32.3, and 34.9 degrees 2θ.

13-14. (canceled)

15. The crystalline form according to claim 1, wherein the crystalline form is characterized by an X-ray powder diffraction (XRPD) pattern comprising diffraction angles, when measured using Cu K_{α} radiation, of about 6.4, 12.7, 14.2, 17.2, 18.9, 20.1, and 21.2 degrees 2θ.

16. The crystalline form according to claim 12, wherein the crystalline form is characterized by an X-ray powder diffraction (XRPD) pattern substantially in accordance with FIG. 9.

17. The crystalline form according to claim 1, wherein the crystalline form is characterized by an X-ray powder diffraction (XRPD) pattern comprising at least three diffraction angles, when measured using Cu K_{α} radiation, selected from a group consisting of about 9.6, 11.0, 11.7, 13.8, 14.3, 15.3, 16.6, 17.2, 17.5, 18.8, 19.3, 20.3, 21.1, 21.4, 22.0, 23.0, 23.6, 24.5, 25.8, 26.2, 27.4, 27.7, 28.6, 29.6, 30.8, 31.0, 31.4, 32.3, 33.3, 35.9, and 39.2 degrees 2θ.

18-19. (canceled)

20. The crystalline form according to claim 1, wherein the crystalline form is characterized by an X-ray powder diffraction (XRPD) pattern comprising diffraction angles, when measured using Cu K_{α} radiation, of about 9.6, 13.8, 20.3, 21.4, 22.0, 24.5, and 26.2 degrees 2θ.

21. The crystalline form according to claim 17, wherein the crystalline form is characterized by an X-ray powder diffraction (XRPD) pattern substantially in accordance with FIG. 12.

22. A pharmaceutical composition comprising the crystalline form according to claim 1 and a pharmaceutically acceptable carrier.

23. The composition according to claim 22 wherein the composition is adapted for oral administration.

24. The composition according to claim 23 wherein the composition is in the form of a tablet or capsule.

25. A method of treating irritable bowel syndrome in a human in need thereof comprising administering to said human an effective amount of the crystalline form according to claim 1.

26. A method of treating irritable bowel syndrome in a human in need thereof comprising administering to said human an effective amount of the composition according to claim 22.

27-30. (canceled)
